(19) United States
(12) Patent Application Publication
(10) Pub. No.: US 2020/0188289 A1
(43) Pub. Date: Jun. 18, 2020

(54) COMPOSITIONS COMPRISING ESSENTIAL OILS

(71) Applicant: Firmenich SA, Geneva 8 (CH)

(72) Inventor: Patrice MERCERET, Geneva (CH)

(73) Assignee: Firmenich SA, Geneva 8 (CH)

(21) Appl. No.: 16/609,357

(22) PCT Filed: May 15, 2018
(86) PCT No.: PCT/IB2018/053393

§ 371 Date: Oct. 29, 2019

Related U.S. Application Data
(60) Provisional application No. 62/506,608, filed on May 15, 2017.

(51) Int. Cl. A61K 8/9783; A61K 8/31; A61K 8/34; A61K 8/35; A61K 8/49; A61K 8/37

(52) U.S. Cl. CPC A61K 8/9783; A61K 8/31; A61K 8/34; A61Q 11/00; A61K 8/4973; A61K 8/37; A61K 8/35

(57) ABSTRACT

The aspects presented herein provide improved flavor compositions, able to deliver long lasting cooling sensation without compromising on the safety profile towards the skin and/or mucosal membrane.

### CROSS REFERENCE TO RELATED APPLICATIONS

This application claims priority to U.S. Provisional Patent Application Ser. No. 62/506,608, filed May 15, 2017, the entire contents of which is hereby incorporated by reference in its entirety.

### FIELD

The present invention concerns the fields of cosmetics, pharmaceuticals, and foodstuffs, in particular preparations having cooling effects on the skin or mucosa, comprising at least one essential oil derived from a hybrid mint plant, together with cooling compounds and/or flavoring.

### BACKGROUND

Compositions of various types have incorporated within them components which provide cooling sensation to mucosal membranes and/or to skin. Such compositions include toothpastes, mouthwashes, perfumes, lotions, shaving creams, post shaving preparations, shampoos, antiperspirants, deodorants, and pharmaceutical products, among others.

The “cooling” effect of menthol is a physiological effect due to the direct action of menthol on the nerve endings of the human body responsive for the detection of hot or cold and is not due to latent heat of evaporation/sublimation. It is believed that the menthol acts as a direct stimulus on the cold receptors at the nerve endings which in turn stimulate the central nervous system.

Although menthol is well established as a physiological cooling agent its use alone, in some compositions, is use may be limited by factors, such as its strong mint odor, or its relative irritant aspect on skin.

Several other compounds have been reported as having an odor or flavor similar to menthol and from time to time have been proposed as flavorants or odorants in a variety of topical and ingestible compositions.

Essential oils obtained from mint plants, such as, for example, peppermint, spearmint and Japanese peppermint, have been widely used in, for example, candies, chewing gums, confectionery, toothpastes, cosmetics and drugs. These essential oils, each of which has a taste with an aroma, a coolness and a refreshing feel, have been used as a flavor.

The characteristics of given essential oil obtained from one variety of mint plant may differ, for example, from (i) an essential oil obtained from another variety of mint plant, or (ii) with the age of the plant, or (iii) over time with storage. In addition, the duration of the coolness and/or refreshing feel may be difficult to control. There have been also significant efforts to provide cooling effect without the strong mint odor typically associated with menthol.

Although there have been these significant efforts to provide enhanced cooling properties to a wide variety of products there is still the need to provide further improved performance.

### SUMMARY

One aspect presented herein, provides a flavor composition comprising:
a. a combination comprising l-carvone and d-limonene,
   wherein the weight ratio of the l-carvone to d-limonene in the combination is from 0.83:1 to 1.25:1; and
b. at least one cooling compound.

In one aspect, the combination is derived from a hybrid mint plant known as 13-S12-2.

Another aspect presented herein, provides a flavor composition comprising:
a. a combination comprising menthofuran and l-menthol, wherein the weight ratio of the menthofuran to l-menthol in the combination is from 0.87:1 to 1.31:1; and
b. at least one cooling compound.

In one aspect, the combination is derived from a hybrid mint plant known as 13-39-9.

Another aspect presented herein, provides a flavor composition comprising:
a. a combination comprising l-menthol and l-menthyl acetate,
   wherein the weight ratio of the l-menthol to l-menthyl acetate in the combination is from 6.15:1 to 9.23:1; and
b. at least one cooling compound.

In one aspect, the combination is derived from a hybrid mint plant known as 13-A36-13.

Another aspect presented herein, provides a flavor composition comprising:
a. a combination comprising l-menthol and l-menthone,
   wherein the weight ratio of the l-menthol to l-menthone in the combination is from 1.66:1 to 2.50:1; and
b. at least one cooling compound.

In one aspect, the combination is derived from a hybrid mint plant known as 07-A3-11.

Another aspect presented herein, provides a flavor composition comprising:
a. a combination comprising l-menthol and l-menthone,
   wherein the weight ratio of the l-menthol to l-menthone in the combination is from 0.98:1 to 1.47:1; and
b. at least one cooling compound.

In one aspect, the combination is derived from a hybrid mint plant known as 09-6-2.

Another aspect presented herein, provides a flavor composition comprising:
a. a combination comprising l-menthol and l-menthone,
   wherein the weight ratio of the l-menthol to l-menthone in the combination is from 2.52:1 to 3.77:1; and
b. at least one cooling compound.

In one aspect, the combination is derived from a hybrid mint plant known as 11-A35-3.

Another aspect presented herein, provides a flavor composition comprising:
a. a combination comprising l-menthol and l-menthone,
   wherein the weight ratio of the l-menthol to l-menthone in the combination is from 1.71:1 to 2.61:1; and
b. at least one cooling compound.

In one aspect, the combination is derived from a hybrid mint plant known as 07-A3-5A.

Another aspect presented herein, provides a flavor composition comprising:
a. a combination comprising l-menthol and l-menthone,
   wherein the weight ratio of the l-menthol to l-menthone in the combination is from 1.73:1 to 2.60:1; and
b. at least one cooling compound.

In one aspect, the combination is derived from a hybrid mint plant known as 14-27-71.

Another aspect presented herein, provides a flavor composition comprising:
a. a combination comprising l-menthol and menthofuran,
   wherein the weight ratio of the l-menthol to menthofuran in the combination is from 1.71:1 to 2.56:1; and
b. at least one cooling compound.

In one aspect, the combination is derived from a hybrid mint plant known as 08-6-10.

Another aspect presented herein, provides a flavor composition comprising:
a. a combination comprising l-menthol and menthofuran,
   wherein the weight ratio of the l-menthol to menthofuran in the combination is from 1.70:1 to 2.55:1; and
b. at least one cooling compound.

In one aspect, the combination is derived from a hybrid mint plant known as 14-41-16.

Another aspect presented herein, provides a flavor composition comprising:
a. a combination comprising l-menthone and l-menthol,
   wherein the weight ratio of the l-menthone to l-menthol in the combination is from 0.95:1 to 1.43:1; and
b. at least one cooling compound.

In one aspect, the combination is derived from a hybrid mint plant known as 08-A20-3.

Another aspect presented herein, provides a flavor composition comprising:
a. a combination comprising l-menthone and l-menthol,
   wherein the weight ratio of the l-menthone to l-menthol in the combination is from 6.17:1 to 9.26:1; and
b. at least one cooling compound.

In one aspect, the combination is derived from a hybrid mint plant known as 14-27-89.

Another aspect presented herein, provides a flavor composition comprising:
a. a combination comprising menthofuran and l-menthyl acetate,
   wherein the weight ratio of the menthofuran to l-menthyl acetate in the combination is from 1.92:1 to 2.89:1; and
b. at least one cooling compound.

In one aspect, the combination is derived from a hybrid mint plant known as 05-19-1.

In one aspect, the amount of the combination present in any one of the flavor compositions presented herein is from 0.1 to 100% by weight.

In one aspect, the combination in any one of the flavor compositions presented herein further comprises at least one extract derived from a commercial mint plant.

In one aspect, the amount of the combination in any one of the flavor compositions presented herein is from 30 to 40% by weight.

In one aspect, the amount of the combination present in any one of the flavor compositions presented herein is from 10 to 40% by weight.

In one aspect, the amount of the combination present in any one of the flavor compositions presented herein is from 10 to 30% by weight.

In one aspect, the amount of the combination present in any one of the flavor compositions presented herein is 35% by weight.

In one aspect, the amount of the combination present in any one of the flavor compositions presented herein is 32% by weight.

In one aspect, the amount of the combination present in any one of the flavor compositions presented herein is 21% by weight.

In one aspect, the at least one cooling compound may be present from 0.005 to 0.4% of any one of the flavor compositions presented herein.

In one aspect, the at least one cooling compound is selected from the group consisting of: WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)—N-(4-Methoxyphenyl)-p-menthanecarboxamide, FEMA 4681; WS-27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1,1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)—N-(4-Methoxyphenyl)-p-menthanecarboxamide (WS-12), FEMA 4681; (2S,5R)—N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680); trans-4-tert-Butylcyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal; (−)-Menthoxypropane-1,2-diol; 3-(1-Menthoxy)-2-methylpropane-1,2-diol, FEMA 3849; Isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3.6,9-trioxadecanoate; (1R,2S,5R)-3-menthyl (2-hydroxyethoxy)acetate; (1R,2S,5R)-menthyl 11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-; menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N—(R)-2-oxotetrahydrofuran-3-yl-(1R,2S,5R)-p-menthane-3-carboxamide; mixture of 2,2,5,6,6-pentamethyl-2,3,6,6a-tetrahydropentalen-3a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA 4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-methan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydroumbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; and combinations thereof.

In one aspect, the at least one cooling compound is 2-(4-methylphenoxy)-N-(1H-pyrazol-3-yl)-N-(2-thienylmethyl) acetamide.

Another aspect presented herein, provides a cosmetic preparation comprising:
a. a flavor composition according to any one of the aspects described herein; and
b. a carrier suitable for cosmetic applications.

In one aspect, the cosmetic preparation is an oral care composition.

Another aspect presented herein, provides a pharmaceutical preparation comprising:
a. a flavor composition according to any one of the aspects described herein; and
b. a carrier suitable for pharmaceutical applications.

Another aspect presented herein, provides a food preparation comprising:
a. a flavor composition according to any one of the aspects described herein; and
b. a carrier suitable for food applications.

Another aspect presented herein, provides a fragrance preparation comprising:
a. a flavor composition according to any one of the aspects described herein; and
b. a carrier suitable for fragrance applications.

### DETAILED DESCRIPTION

In the following description, reference is made to specific embodiments which may be practiced, which is shown by way of illustration. These embodiments are described in detail to enable those skilled in the art to practice the invention described herein, and it is to be understood that other embodiments may be utilized and that logical changes may be made without departing from the scope of the aspects presented herein. The following description of example embodiments is, therefore, not to be taken in a limited sense, and the scope of the various aspects presented herein is defined by the appended claims.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

One aspect presented herein provides a flavor composition comprising:
a. a combination selected from the group consisting of:
   i. a combination comprising l-carvone and d-limonene, wherein the weight ratio of the l-carvone to d-limonene in the combination is from 0.83:1 to 1.25:1;
   ii. a combination comprising menthofuran and l-menthol, wherein the weight ratio of the menthofuran to l-menthol in the combination is from 0.87:1 to 1.31:1;
   iii. a combination comprising l-menthol and l-menthyl acetate, wherein the weight ratio of the l-menthol to l-menthyl acetate in the combination is from 6.15:1 to 9.23:1;
   iv. a combination comprising l-menthol and l-menthone, wherein the weight ratio of the l-menthol to l-menthone in the combination is from 1.66:1 to 2.50:1;
   v. a combination comprising l-menthol and l-menthone, wherein the weight ratio of the l-menthol to l-menthone in the combination is from 0.98:1 to 1.47:1;
   vi. a combination comprising l-menthol and l-menthone, wherein the weight ratio of the l-menthol to l-menthone in the combination is from 2.52:1 to 3.77:1;
   vii. a combination comprising l-menthol and l-menthone, wherein the weight ratio of the l-menthol to l-menthone in the combination is from 1.71:1 to 2.61:1;
   viii. a combination comprising l-menthol and l-menthone, wherein the weight ratio of the l-menthol to l-menthone in the combination is from 1.73:1 to 2.60:1;
   ix. a combination comprising l-menthol and menthofuran, wherein the weight ratio of the l-menthol to menthofuran in the combination is from 1.71:1 to 2.56:1;
   x. a combination comprising l-menthol and menthofuran, wherein the weight ratio of the l-menthol to menthofuran in the combination is from 1.70:1 to 2.55:1;
   xi. a combination comprising l-menthone and l-menthol, wherein the weight ratio of the l-menthone to l-menthol in the combination is from 0.95:1 to 1.43:1;
   xii. a combination comprising l-menthone and l-menthol, wherein the weight ratio of the l-menthone to l-menthol in the combination is from 6.17:1 to 9.26:1;
   xiii. a combination comprising menthofuran and l-menthyl acetate, wherein the weight ratio of the menthofuran to l-menthyl acetate in the combination is from 1.92:1 to 2.89:1; and
b. at least one cooling compound.

In one aspect, the amount of the combination present in the flavor composition is from 0.1 to 100% by weight. Alternatively, in one aspect, the amount of the combination present in the flavor composition is from 10 to 30% by weight. Alternatively, in on aspect, the amount of the combination present in the flavor composition is from 30 to 40% by weight. Alternatively, in one aspect, the amount of the combination present in the flavor composition is 35% by weight. Alternatively, in one aspect, the amount of the combination present in the flavor composition is 32% by weight. Alternatively, in one aspect, the amount of the combination present in the flavor composition is 21% by weight. Alternatively, in one aspect, the amount of the combination present in the flavor composition is from 10 to 40% by weight.

In one aspect, the flavor composition further comprises at least one extract derived from a commercial mint plant.

In one aspect, the amount of the at least one cooling compound present in the flavor composition is from 0.005 to 0.4% by weight. In one aspect, the at least one cooling compound is selected from the group consisting of: WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)—N-(4-Methoxyphenyl)-p-menthanecarboxamide, FEMA 4681; WS-27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1,1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)—N-(4-Methoxyphenyl)-p-menthanecarboxamide (WS-12), FEMA 4681; (2S,5R)—N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy]ethanol, FEMA 4718; (2,6-Diethyl-5-isopropyl-2-methyltetrahydropyran, FEMA 4680); trans-4-tert-Butylcyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal; (−)-Menthoxypropane-1,2-diol; 3-(1-Menthoxy)-2-methylpropane-1,2-diol, FEMA 3849; Isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3.6,9-trioxadecanoate; (1R,2S,5R)-3-menthyl (2-hydroxyethoxy)acetate; (1R,2S,5R)-menthyl 11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-; menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N—(R)-2-oxotetrahydrofuran-3-yl-(1R,2S,5R)-p-menthane-3-carboxamide; mixture of 2,2,5,6,6-pentamethyl-2,3,6,6a-tetrahydropentalen-3a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA 4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-methan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydroumbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; and combinations thereof. Alternatively, in one aspect, the at least one cooling compound is 2-(4-methylphenoxy)-N-(1H-pyrazol-3-yl)-N-(2-thienylmethyl) acetamide.

One aspect presented herein provides a cosmetic preparation comprising:
a. the flavor composition according to the aspects presented herein; and
b. a carrier suitable for cosmetic applications.

One aspect presented herein provides a pharmaceutical preparation comprising:
a. the flavor composition according to the aspects presented herein; and
b. a carrier suitable for pharmaceutical applications.

One aspect presented herein provides a food preparation comprising:
a. the flavor composition according to the aspects presented herein; and
b. a carrier suitable for food applications.

One aspect presented herein provides a fragrance preparation comprising:
a. the flavor composition according to the aspects presented herein; and
b. a carrier suitable for fragrance applications.

### The Flavor Compositions.

One aspect presented herein, provides improved flavor compositions, able to deliver long lasting cooling sensation without compromising on the safety profile towards the skin and/or mucosal membrane or even improving it. A further aspect provided herein, delivers such benefits with an improved odor profile. The preparations presented herein may be used in cosmetics, pharmaceuticals, foodstuffs, fragrances, air care products, and the like.

Without intending to be limited to any particular theory, the desired cooling properties may be imparted by the particular aromatic compounds, and/or the relative amounts of the particular aromatic compounds present in the combination.

Moreover, desired cooling properties may be imparted by blending several combinations, thereby giving rise to a specific mixture of aromatic compounds and/or different relative amounts of the particular aromatic compounds.

One aspect presented herein, provides a flavor composition comprising:
a. a combination comprising l-carvone and d-limonene,
   wherein the weight ratio of the l-carvone to d-limonene in the combination is from 0.83:1 to 1.25:1; and
   b. at least one cooling compound.

In one aspect, the combination is derived from a hybrid mint plant known as 13-S12-2.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-carvone and d-limonene in the combination is from 5 to 7% by weight.

In one aspect, the amount of the combination present in the flavor composition is 35% by weight.

In one aspect, the total amount of the l-carvone and d-limonene in the combination is 7% by weight.

In one aspect, the amount of the combination present in the flavor composition is from 10 to 30% by weight.

In one aspect, the total amount of the l-carvone and d-limonene in the combination is from 50 to 70% by weight.

In one aspect, the amount of the combination present in the flavor composition is 21% by weight.

In one aspect, the total amount of the l-carvone and d-limonene in the combination is 66% by weight.

In one aspect, the amount of the combination present in the flavor composition is from 10 to 40% by weight.

In one aspect, the total amount of the l-carvone and d-limonene in the combination is from 2 to 5% by weight.

In one aspect, the amount of the combination present in the flavor composition is 32% by weight.

In one aspect, the total amount of the l-carvone and d-limonene in the combination is 3% by weight.

One aspect presented herein, provides a flavor composition comprising:
a. a combination comprising menthofuran and l-menthol,
   wherein the weight ratio of the menthofuran to l-menthol in the combination is from 0.87:1 to 1.31:1; and
b. at least one cooling compound.

In one aspect, the combination is derived from a hybrid mint plant known as 13-39-9.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the menthofuran and l-menthol in the combination is from 5 to 7% by weight.

In one aspect, the amount of the combination present in the flavor composition is 35% by weight.

In one aspect, the total amount of the menthofuran and l-menthol in the combination is 7% by weight.

In one aspect, the amount of the combination present in the flavor composition is from 10 to 30% by weight.

In one aspect, the total amount of the menthofuran and l-menthol in the combination is from 50 to 70% by weight.

In one aspect, the amount of the combination present in the flavor composition is 21% by weight.

In one aspect, the total amount of the menthofuran and l-menthol in the combination is 66% by weight.

In one aspect, the amount of the combination present in the flavor composition is from 10 to 40% by weight.

In one aspect, the total amount of the menthofuran and l-menthol in the combination is from 2 to 5% by weight.

In one aspect, the amount of the combination present in the flavor composition is 32% by weight.

In one aspect, the total amount of the menthofuran and l-menthol in the combination is 3% by weight.

One aspect presented herein, provides a flavor composition comprising:
a. a combination comprising l-menthol and l-menthyl acetate,
   wherein the weight ratio of the l-menthol to l-menthyl acetate in the combination is from 6.15:1 to 9.23:1; and
b. at least one cooling compound.

In one aspect, the combination is derived from a hybrid mint plant known as 13-A36-13.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthyl acetate in the combination is from 5 to 7% by weight.

In one aspect, the amount of the combination present in the flavor composition is 35%.

In one aspect, the total amount of the l-menthol and l-menthyl acetate in the combination is 7% by weight.

In one aspect, the amount of the combination present in the flavor composition is from 10 to 30% by weight.

In one aspect, the total amount of the l-menthol and l-menthyl acetate in the combination is from 50 to 70% by weight.

In one aspect, the amount of the combination present in the flavor composition is 21%.

In one aspect, the total amount of the l-menthol and l-menthyl acetate in the combination is 66% by weight.

In one aspect, the amount of the combination present in the flavor composition is from 10 to 40% by weight.

In one aspect, the total amount of the l-menthol and l-menthyl acetate in the combination is from 2 to 5% by weight.

In one aspect, the amount of the combination present in the flavor composition is 32% by weight.

In one aspect, the total amount of the l-menthol and l-menthyl acetate in the combination is 3% by weight.

One aspect presented herein, provides a flavor composition comprising:
a. a combination comprising l-menthol and l-menthone,
   wherein the weight ratio of the l-menthol to l-menthone in the combination is from 1.66:1 to 2.50:1; and
b. at least one cooling compound.

In one aspect, the combination is derived from a hybrid mint plant known as 07-A3-11.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthone in the combination is from 5 to 7% by weight.

In one aspect, the amount of the combination present in the flavor composition is 35% by weight.

In one aspect, the total amount of the l-menthol and l-menthone in the combination is 7% by weight.

In one aspect, the amount of the combination present in the flavor composition is from 10 to 30% by weight.

In one aspect, the total amount of the l-menthol and l-menthone in the combination is from 50 to 70% by weight.

In one aspect, the amount of the combination present in the flavor composition is 21% by weight.

In one aspect, the total amount of the l-menthol and l-menthone in the combination is 66% by weight.

In one aspect, the amount of the combination present in the flavor composition is from 10 to 40% by weight.

In one aspect, the total amount of the l-menthol and l-menthone in the combination is from 2 to 5% by weight.

In one aspect, the amount of the combination present in the flavor composition is 32% by weight.

In one aspect, the total amount of the l-menthol and l-menthone in the combination is 3% by weight.

One aspect presented herein, provides a flavor composition comprising:
a. a combination comprising l-menthol and l-menthone,
   wherein the weight ratio of the l-menthol to l-menthone in the combination is from 0.98:1 to 1.47:1; and
b. at least one cooling compound.

In one aspect, the combination is derived from a hybrid mint plant known as 09-6-2.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthone in the combination is from 5 to 7% by weight.

In one aspect, the amount of the combination present in the flavor composition is 35% by weight.

In one aspect, the total amount of the l-menthol and l-menthone in the combination is 7% by weight.

In one aspect, the amount of the combination present in the flavor composition is from 10 to 30% by weight.

In one aspect, the total amount of the l-menthol and l-menthone in the combination is from 50 to 70% by weight.

In one aspect, the amount of the combination present in the flavor composition is 21% by weight.

In one aspect, the total amount of the l-menthol and l-menthone in the combination is 66% by weight.

In one aspect, the amount of the combination present in the flavor composition is from 10 to 40% by weight.

In one aspect, the total amount of the l-menthol and l-menthone in the combination is from 2 to 5% by weight.

In one aspect, the amount of the combination present in the flavor composition is 32% by weight.

In one aspect, the total amount of the l-menthol and l-menthone in the combination is 3% by weight.

One aspect presented herein, provides a flavor composition comprising:
a. a combination comprising l-menthol and l-menthone,
   wherein the weight ratio of the l-menthol to l-menthone in the combination is from 2.52:1 to 3.77:1; and
b. at least one cooling compound.

In one aspect, the combination is derived from a hybrid mint plant known as 11-A35-3.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthone in the combination is from 5 to 7% by weight by weight.

In one aspect, the amount of the combination present in the flavor composition is 35% by weight.

In one aspect, the total amount of the l-menthol and l-menthone in the combination is 7% by weight.

In one aspect, the amount of the combination present in the flavor composition is from 10 to 30% by weight.

In one aspect, the total amount of the l-menthol and l-menthone in the combination is from 50 to 70% by weight.

In one aspect, the amount of the combination present in the flavor composition is 21% by weight.

In one aspect, the total amount of the l-menthol and l-menthone in the combination is 66% by weight.

In one aspect, the amount of the combination present in the flavor composition is from 10 to 40% by weight.

In one aspect, the total amount of the l-menthol and l-menthone in the combination is from 2 to 5% by weight.

In one aspect, the amount of the combination present in the flavor composition is 32% by weight.

In one aspect, the total amount of the l-menthol and l-menthone in the combination is 3% by weight.

One aspect presented herein, provides a flavor composition comprising:
a. a combination comprising l-menthol and l-menthone,
   wherein the weight ratio of the l-menthol to l-menthone in the combination is from 1.71:1 to 2.61:1; and
b. at least one cooling compound.

In one aspect, the combination is derived from a hybrid mint plant known as 07-A3-5A.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthone in the combination is from 5 to 7% by weight.

In one aspect, the amount of the combination present in the flavor composition is 35% by weight.

In one aspect, the total amount of the l-menthol and l-menthone in the combination is 7% by weight.

In one aspect, the amount of the combination present in the flavor composition is from 10 to 30% by weight.

In one aspect, the total amount of the l-menthol and l-menthone in the combination is from 50 to 70% by weight.

In one aspect, the amount of the combination present in the flavor composition is 21% by weight.

In one aspect, the total amount of the l-menthol and l-menthone in the combination is 66% by weight.

In one aspect, the amount of the combination present in the flavor composition is from 10 to 40%.

In one aspect, the total amount of the l-menthol and l-menthone in the combination is from 2 to 5% by weight.

In one aspect, the amount of the combination present in the flavor composition is 32% by weight.

In one aspect, the total amount of the l-menthol and l-menthone in the combination is 3% by weight.

One aspect presented herein, provides a flavor composition comprising:
a. a combination comprising l-menthol and l-menthone,
   wherein the weight ratio of the l-menthol to l-menthone in the combination is from 1.73:1 to 2.60:1; and
b. at least one cooling compound.

In one aspect, the combination is derived from a hybrid mint plant known as 14-27-71.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and l-menthone in the combination is from 5 to 7% by weight.

In one aspect, the amount of the combination present in the flavor composition is 35% by weight.

In one aspect, the total amount of the l-menthol and l-menthone in the combination is 7% by weight.

In one aspect, the amount of the combination present in the flavor composition is from 10 to 30% by weight.

In one aspect, the total amount of the l-menthol and l-menthone in the combination is from 50 to 70% by weight.

In one aspect, the amount of the combination present in the flavor composition is 21%.

In one aspect, the total amount of the l-menthol and l-menthone in the combination is 66% by weight.

In one aspect, the amount of the combination present in the flavor composition is from 10 to 40% by weight.

In one aspect, the total amount of the l-menthol and l-menthone in the combination is from 2 to 5% by weight.

In one aspect, the amount of the combination present in the flavor composition is 32% by weight.

In one aspect, the total amount of the l-menthol and l-menthone in the combination is 3% by weight.

One aspect presented herein, provides a flavor composition comprising:
a. a combination comprising l-menthol and menthofuran,
   wherein the weight ratio of the l-menthol to menthofuran in the combination is from 1.71:1 to 2.56:1; and
b. at least one cooling compound.

In one aspect, the combination is derived from a hybrid mint plant known as 08-6-10.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and menthofuran in the combination is from 5 to 7% by weight.

In one aspect, the amount of the combination present in the flavor composition is 35% by weight.

In one aspect, the total amount of the l-menthol and menthofuran in the combination is 7% by weight.

In one aspect, the amount of the combination present in the flavor composition is from 10 to 30% by weight.

In one aspect, the total amount of the l-menthol and menthofuran in the combination is from 50 to 70% by weight.

In one aspect, the amount of the combination present in the flavor composition is 21% by weight.

In one aspect, the total amount of the l-menthol and menthofuran in the combination is 66% by weight.

In one aspect, the amount of the combination present in the flavor composition is from 10 to 40% by weight.

In one aspect, the total amount of the l-menthol and menthofuran in the combination is from 2 to 5% by weight.

In one aspect, the amount of the combination present in the flavor composition is 32% by weight.

In one aspect, the total amount of the l-menthol and menthofuran in the combination is 3% by weight.

One aspect presented herein, provides a flavor composition comprising:
a. a combination comprising l-menthol and menthofuran,
   wherein the weight ratio of the l-menthol to menthofuran in the combination is from 1.70:1 to 2.55:1; and
b. at least one cooling compound.

In one aspect, the combination is derived from a hybrid mint plant known as 14-41-16.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthol and menthofuran in the combination is from 5 to 7% by weight.

In one aspect, the amount of the combination present in the flavor composition is 35% by weight.

In one aspect, the total amount of the l-menthol and menthofuran in the combination is 7% by weight.

In one aspect, the amount of the combination present in the flavor composition is from 10 to 30% by weight.

In one aspect, the total amount of the l-menthol and menthofuran in the combination is from 50 to 70% by weight.

In one aspect, the amount of the combination present in the flavor composition is 21% by weight.

In one aspect, the total amount of the l-menthol and menthofuran in the combination is 66% by weight.

In one aspect, the amount of the combination present in the flavor composition is from 10 to 40% by weight.

In one aspect, the total amount of the l-menthol and menthofuran in the combination is from 2 to 5% by weight.

In one aspect, the amount of the combination present in the flavor composition is 32% by weight.

In one aspect, the total amount of the l-menthol and menthofuran in the combination is 3% by weight.

One aspect presented herein, provides a flavor composition comprising:
a. a combination comprising l-menthone and l-menthol,
   wherein the weight ratio of the l-menthone to l-menthol in the combination is from 0.95:1 to 1.43:1; and
b. at least one cooling compound.

In one aspect, the combination is derived from a hybrid mint plant known as 08-A20-3.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the l-menthone and l-menthol in the combination is from 5 to 7% by weight.

In one aspect, the amount of the combination present in the flavor composition is 35% by weight.

In one aspect, the total amount of the l-menthone and l-menthol in the combination is 7% by weight.

In one aspect, the amount of the combination present in the flavor composition is from 10 to 30% by weight.

In one aspect, the total amount of the l-menthone and l-menthol in the combination is from 50 to 70% by weight.

In one aspect, the amount of the combination present in the flavor composition is 21% by weight.

In one aspect, the total amount of the l-menthone and l-menthol in the combination is 66% by weight.

In one aspect, the amount of the combination present in the flavor composition is from 10 to 40% by weight.

In one aspect, the total amount of the l-menthone and l-menthol in the combination is from 2 to 5% by weight.

In one aspect, the amount of the combination present in the flavor composition is 32% by weight.

In one aspect, the total amount of the l-menthone and l-menthol in the combination is 3% by weight.

One aspect presented herein, provides a flavor composition comprising:
a. a combination comprising l-menthone and l-menthol,
   wherein the weight ratio of the l-menthone to l-menthol in the combination is from 6.17:1 to 9.26:1; and
b. at least one cooling compound.

In one aspect, the combination is derived from a hybrid mint plant known as 14-27-89.

In one aspect, the amount of the combination present in the flavor composition is from 0.1 to 100%.

In one aspect, the combination further comprises at least one extract derived from a commercial mint plant.

In one aspect, the amount of the combination present in the flavor composition is from 30 to 40% by weight.

In one aspect, the total amount of the l-menthone and l-menthol in the combination is from 5 to 7% by weight.

In one aspect, the amount of the combination present in the flavor composition is 35% by weight.

In one aspect, the total amount of the l-menthone and l-menthol in the combination is 7% by weight.

In one aspect, the amount of the combination present in the flavor composition is from 10 to 30% by weight.

In one aspect, the total amount of the l-menthone and l-menthol in the combination is from 50 to 70% by weight.

In one aspect, the amount of the combination present in the flavor composition is 21% by weight.

In one aspect, the total amount of the l-menthone and l-menthol in the combination is 66% by weight.

In one aspect, the amount of the combination present in the flavor composition is from 10 to 40% by weight.

In one aspect, the total amount of the l-menthone and l-menthol in the combination is from 2 to 5% by weight.

In one aspect, the amount of the combination present in the flavor composition is 32% by weight.

In one aspect, the total amount of the l-menthone and l-menthol in the combination is 3% by weight.

One aspect presented herein, provides a flavor composition comprising:
a. a combination comprising menthofuran and l-menthyl acetate,
   wherein the weight ratio of the menthofuran to l-menthyl acetate in the combination is from 1.92:1 to 2.89:1; and
b. at least one cooling compound.

In one aspect, the combination is derived from a hybrid mint plant known as 05-19-1.

In one aspect, the amount of the combination present in the flavor composition is from 0.1 to 100% by weight.

In one aspect, the combination further comprises at least one extract derived from a commercial mint plant.

In one aspect, the amount of the combination present in the flavor composition is from 30 to 40% by weight.

In one aspect, the total amount of the menthofuran and l-menthyl acetate in the combination is from 5 to 7% by weight.

In one aspect, the amount of the combination present in the flavor composition is 35% by weight.

In one aspect, the total amount of the menthofuran and l-menthyl acetate in the combination is 7% by weight.

In one aspect, the amount of the combination present in the flavor composition is from 10 to 30% by weight.

In one aspect, the total amount of the menthofuran and l-menthyl acetate in the combination is from 50 to 70% by weight.

In one aspect, the amount of the combination present in the flavor composition is 21%.

In one aspect, the total amount of the menthofuran and l-menthyl acetate in the combination is 66% by weight.

In one aspect, the amount of the combination present in the flavor composition is from 10 to 40% by weight.

In one aspect, the total amount of the menthofuran and l-menthyl acetate in the combination is from 2 to 5% by weight.

In one aspect, the amount of the combination present in the flavor composition is 32%.

In one aspect, the total amount of the menthofuran and l-menthyl acetate in the combination is 3% by weight.

In one aspect, the at least one cooling compound may be present from 0.005 to 0.4% of the flavor composition. In one aspect, the at least one cooling compound is present at 0.4%, alternatively, at 0.3%, alternatively, at 0.2%, alternatively, at 0.1%, alternatively, at 0.05%, alternatively, at 0.04%, alternatively, at 0.03%, alternatively, at 0.02%, alternatively, at 0.01%, alternatively, at 0.009%, alternatively, at 0.008%, alternatively, at 0.007%, alternatively, at 0.006%, alternatively, at 0.005%.

The amount of the combination present in the flavor composition may range from 0.1 to 100%. For example, the amount of the combination may be 90%, or alternatively, 80%, or alternatively, 70%, or alternatively, 60%, or alternatively, 50%, or alternatively, 40%, or alternatively, 30%, or alternatively, 20%, or alternatively, 10%, or alternatively, 9%, or alternatively, 8%, or alternatively, 7%, or alternatively, 6%, or alternatively, 5%, or alternatively, 4%, or alternatively, 3%, or alternatively, 2%, or alternatively, 1%, or alternatively, 0.5%, or alternatively, 0.4%, or alternatively, 0.3%, or alternatively, 0.2%, or alternatively, 0.1%.

In one aspect, the combination further comprises at least one extract derived from a commercial mint plant.

In aspects where the combination further comprises at least one extract derived from a commercial mint plant, the portion of the combination that is not derived from a commercial mint plant may range from 0.1 to 100% of the combination. For example, the portion of the combination that is not derived from a commercial mint plant may be 90%, or alternatively, 80%, or alternatively, 70%, or alternatively, 60%, or alternatively, 50%, or alternatively, 40%, or alternatively, 30%, or alternatively, 20%, or alternatively, 10%, or alternatively, 9%, or alternatively, 8%, or alternatively, 7%, or alternatively, 6%, or alternatively, 5%, or alternatively, 4%, or alternatively, 3%, or alternatively, 2%, or alternatively, 1%, or alternatively, 0.5%, or alternatively, 0.4%, or alternatively, 0.3%, or alternatively, 0.2%, or alternatively, 0.1% of the combination.

In one aspect, the amount of the combination present in the flavor composition is from 30 to 40%, wherein from 5 to 7% of the combination is not derived from a commercial mint plant.

In one aspect, the flavor composition comprises from 35% plant extract, wherein 7% of the combination is not derived from a commercial mint plant.

In one aspect, the combination comprises 1 to 4% l-limonene, 0.5 to 7% 1,8-cineole, 2 to 62% l-menthone, 0 to 50% menthofuran, 0.5 to 5% isomenthone, 0.5 to 20% 1-menthyl acetate, 1 to 4% neo-menthol, 7 to 65% l-menthol, and 0 to 2% pulegone, wherein the percentage is determined by calculation of the relative peak areas of a gas chromatograph of the combination.

In one aspect, the combination further comprises 0.5 to 2% β-caryophyllene.

In one aspect, the combination further comprises 0.5 to 2% germacrene-D.

In one aspect, the combination comprises 0.5 to 2% a-pinene, 1 to 2% β-pinene, 1 to 4% l-limonene, 0.5 to 7% 1,8-cineole, 2 to 62% l-menthone, 0 to 50% menthofuran, 0.5 to 5% isomenthone, 0.5 to 20% l-menthyl acetate, 1 to 4% neo-menthol, 7 to 65% 1-menthol, and 0 to 2% pulegone, wherein the percentage is determined by calculation of the relative peak areas of a gas chromatograph of the combination.

In one aspect, the combination further comprises 0.5 to 2% β-caryophyllene.

In one aspect, the combination comprises 20 to 40% d-limonene, 0.5 to 7% 1,8-cineole, 1 to 3% 3-octanol, 1 to 3% menthone, 1 to 4% dihydrocarvone, and 20 to 40% l-carvone.

In certain aspects, the desired cooling properties are imparted to the flavor composition by using essential oils, or combinations thereof, thereby giving rise to a specific mixture of aromatic compounds and/or different relative amounts of the particular aromatic compounds.

In certain aspects, the flavor composition comprises an essential oil derived from a 07-A3-5A hybrid mint plant, and a taste-masking agent, and at least one flavoring agent. Examples of taste-masking agents and flavoring agents are disclosed in U.S. Patent Application Publication No. 2013/0330282.

In certain aspects, the flavor composition further comprises a food grade solvent. Examples of food grade solvents include, but are not limited to vegetable oils, polyethylene glycol, triacetine, and the like.

Examples of vegetable oils suitable for use in the present invention include, but are not limited to linseed oil, coconut oil, corn oil, sunflower oil, and the like.

In certain aspects, the flavor composition further comprises an additional flavor ingredient, such as, for example, a menthol, a cooling compound, and the like. Bu way of illustration, but not intended to be limiting, flavor formulations according to certain aspects of the present invention are presented below.

In one aspect, the flavor composition comprises anethol, carvone gauche, the cooling compound known as Freeze 10 ethyl acetate, menthol melted, menthol racemic, terpeneless essential oil derived from a M arvensis mint plant, an essential oil derived from a 07-A3-5A hybrid mint plant, an essential oil derived from a Mint Piperita Rectified India commercial mint plant, an essential oil derived from a Spicata Native commercial mint plant, an essential oil derived from a Spicata US commercial mint plant, and linseed oil.

In one aspect, the anethol is present from 5 to 15% of the flavor composition, for example, at 5%, or 6%, or 7%, or 8%, or 9%, or 10%, or 11%, or 12%, or 13%, or 14%, or 15%. In one aspect, the anethol is present at 9.3% of the flavor composition.

In one aspect, the carvone gauche is present from 5 to 15% of the flavor composition, for example, at 5%, or 6%, or 7%, or 8%, or 9%, or 10%, or 11%, or 12%, or 13%, or 14%, or 15%. In one aspect, the carvone gauche is present at 8.8% of the flavor composition.

In one aspect, the cooling compound known as Freeze 10 ethyl acetate is present from 0.5 to 10% of the flavor composition, for example, at 0.5%, or 0.6%, or 0.7%, or 0.8%, or 0.9%, or 1%, or 2%, or 3%, or 4%, or 5%, or 6%, or 7%, or 8%, or 9%, or 10%. In one aspect, the cooling compound known as Freeze 10 ethyl acetate is present at 0.9% of the flavor composition.

In one aspect, the menthol melted is present from 20 to 40% of the flavor composition, for example, at 20%, or 25%, or 30%, or 35%, or 40%. In one aspect, the menthol melted is present at 27.3% of the flavor composition.

In one aspect, the menthol racemic is present from 4 to 7% of the flavor composition, for example, at 4%, or 5%, or 6%, or 7%, of composition (a). In one aspect, the menthol racemic is present at 5.5% of the flavor composition.

In one aspect, the terpeneless essential oil derived from a M arvensis mint plant is present from 4 to 7% of the flavor composition, for example, at 4%, or 5%, or 6%, or 7%. In one aspect, the terpeneless essential oil derived from a M arvensis mint plant is present at 6.5% of the flavor composition.

In one aspect, the essential oil derived from a 07-A3-5A hybrid mint plant is present from 1 to 2.5% of the flavor composition, for example, at 1%, or 1.5%, or 2%, or 2.5%. In one aspect, the essential oil derived from a 07-A3-5A hybrid mint plant is present at 2.5% of the flavor composition.

In one aspect, the essential oil derived from a Mint Piperita Rectified India commercial mint plant is present from 4 to 7% of the flavor composition, for example, at 4%, or 5%, or 6%, or 7%. In one aspect, the essential oil derived from a Mint Piperita Rectified India commercial mint plant is present at 6.8% of the flavor composition.

In one aspect, the essential oil derived from a Spicata Native commercial mint plant is present from 8 to 15% of the flavor composition, for example, at 8%, or 9%, or 10%, or 11%, or 12%, or 13%, or 14%, or 15%. In one aspect, the essential oil derived from a Spicata Native commercial mint plant is present at 9.1% of the flavor composition.

In one aspect, the essential oil derived from a Spicata US commercial mint plant is present from 8 to 15% of the flavor composition, for example, at 8%, or 9%, or 10%, or 11%, or 12%, or 13%, or 14%, or 15%. In one aspect, the essential oil derived from a Spicata US commercial mint plant is present at 10.4% of the flavor composition.

In one aspect, the linseed oil is present from 8 to 15% of the flavor composition, for example, at 8%, or 9%, or 10%, or 11%, or 12%, or 13%, or 14%, or 15%. In one aspect, the linseed oil is present at 12.9% of the flavor composition.

In one aspect, the flavor composition is described in Table 19.

In one aspect, the flavor composition comprises from 10 to 30% of the combination, wherein from 50 to 70% of the combination is not derived from a hybrid mint plant.

In one aspect, the flavor composition comprises 21% of the combination, wherein 66% of the combination is not derived from a hybrid mint plant.

In one aspect, the flavor composition comprises Nova Mint Flavor, Freeze Bamboo Mint Flavor, anethol, decal, eucalyptol, eucalyptus globulus, eugenol, the cooling compound known as Freeze 10 ethyl acetate, HEDIONE FAB @ 10% Menth Bresil, allyle heptanoate, Menthe Bresil, menthol melted, menthol racemic, an essential oil derived from a Euromint commercial mint plant, an essential oil derived from a 07-A3-5A hybrid mint plant, an essential oil derived from a Mint Piperita Cascade commercial mint plant, octalactone G, violette A1 II @ 1% of a terpeneless essential oil derived from a M arvensis mint plant.

In one aspect, the Nova Mint Flavor is present from 0.5 to 1.5% of the flavor composition, for example, at 0.5%, or 0.6%, or 0.7%, or 0.8%, or 0.9%, or 1.0%, or 1.1%, or 1.2%, or 1.3%, or 1.4%, or 1.5%. In one aspect, the Nova Mint Flavor is present at 1.2% of the flavor composition.

In one aspect, the Freeze Bamboo Mint is present from 2 to 5% of the flavor composition, for example, at 2%, or 2.5%, or 3%, or 3.5%, or 4%, or 4.5%, or 5%. In one aspect, the Freeze Bamboo Mint is present at 4% of the flavor composition.

In one aspect, the anethol is present from 5 to 15% of the flavor composition, for example, at 5%, or 6%, or 7%, or 8%, or 9%, or 10%, or 11%, or 12%, or 13%, or 14%, or 15%. In one aspect, the anethol is present at 9.11% of the flavor composition.

In one aspect, the decal is present from 0.05 to 0.5% of the flavor composition, for example, at 0.05%, or 0.1%, or 0.15%, or 0.2%, or 0.25%, or 0.3%, or 0.35%, or 0.4%, or 0.45%, or 0.5%. In one aspect, the decal is present at 0.2% of the flavor composition.

In one aspect, the eucalyptol is present from 1 to 2.5% of the flavor composition, for example, at 1%, or 1.5%, or 2%, or 2.5%. In one aspect, eucalyptol is present at 1.55% of the flavor composition.

In one aspect, the eucalyptus globulus is present from 1 to 3.5% of the flavor composition, for example, at 1%, or 1.5%, or 2%, or 2.5%, or 3%, or 3.5%. In one aspect, eucalyptus globulus is present at 2.5% of the flavor composition.

In one aspect, the eugenol is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the eugenol is present at 0.84% of the flavor composition.

In one aspect, the cooling compound known as Freeze 10 ethyl acetate is present from 0.5 to 10% of the flavor composition, for example, at 0.5%, or 0.6%, or 0.7%, or 0.8%, or 0.9%, or 1%, or 2%, or 3%, or 4%, or 5%, or 6%, or 7%, or 8%, or 9%, or 10%. In one aspect, the cooling compound known as Freeze 10 ethyl acetate is present at 1.48% of the flavor composition.

In one aspect, the HEDIONE FAB @ 10% Menth Bresil is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the HEDIONE FAB @ 10% Menth Bresil is present at 0.1% of the flavor composition.

In one aspect, the allyle heptanoate is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the allyle heptanoate is present at 0.3% of the flavor composition.

In one aspect, the cooling compound known as Menthe Bresil is present from 0.5 to 10% of the flavor composition, for example, at 0.5%, or 0.6%, or 0.7%, or 0.8%, or 0.9%, or 1%, or 2%, or 3%, or 4%, or 5%, or 6%, or 7%, or 8%, or 9%, or 10%. In one aspect, the Menthe Bresil is present at 8.08% of the flavor composition.

In one aspect, the menthol melted is present from 20 to 50% of the flavor composition, for example, at 20%, or 25%, or 30%, or 35%, or 40%, or 50%. In one aspect, the menthol melted is present at 41.08% of the flavor composition.

In one aspect, the menthol racemic is present from 4 to 8% of the flavor composition, for example, at 4%, or 5%, or 6%, or 7%, or 8%, of composition (a). In one aspect, the menthol racemic is present at 7.3% of the flavor composition.

In one aspect, the essential oil derived from a Euromint commercial mint plant is present from 0.5 to 2.5% of the flavor composition, for example, at 0.5%, or 1%, or 1.5%, or 2%, or 2.5%. In one aspect, the essential oil derived from a Euromint commercial mint plant is present at 0.95% of the flavor composition.

In one aspect, the essential oil derived from a 07-A3-5A hybrid mint plant is present from 10 to 25% of the flavor composition, for example, at 10%, or 15%, or 20%, or 25%. In one aspect, the essential oil derived from a 07-A3-5A hybrid mint plant is present at 14.2% of the flavor composition.

In one aspect, the essential oil derived from a Mint Piperita Cascade commercial mint plant is present from 4 to 7% of the flavor composition, for example, at 4%, or 5%, or 6%, or 7%. In one aspect, the essential oil derived from a Mint Piperita Cascade commercial mint plant is present at 6.25% of the flavor composition.

In one aspect, the octalactone G is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the octalactone G is present at 0.48% of the flavor composition.

In one aspect, the violette A1 II @ 1% of a terpeneless essential oil derived from a M arvensis mint plant is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the violette A1 II @ 1% of a terpeneless essential oil derived from a M arvensis mint plant is present at 0.48% of the flavor composition.

In one aspect, the flavor composition is described in Table 20.

In one aspect, the flavor composition comprises from 10 to 40% of the combination, wherein from 2 to 5% of the combination is not derived from a hybrid mint plant.

In one aspect, the flavor composition comprises 32% plant extract, wherein 3% of the combination is not derived from a hybrid mint plant.

In one aspect, the flavor composition comprises Rainforest Mint flavor, anethol, eucalyptus globulus, eugenol F, the cooling compound known as Freeze 10 ethyl acetate, glycyrrhizine ammoniacale @ 1% PG, menthol melted, menthol racemic, an essential oil derived from a M arvensis TPC commercial mint plant, an essential oil derived from a 07-A3-5A hybrid mint plant, an essential oil derived from a Euromint commercial mint plant, an essential oil derived from a Piperita ABS commercial mint plant @ 10% Mint Piperita Rectified India commercial mint plant, an essential oil derived from a Mint Piperita Cascade commercial mint plant, an essential oil derived from a Mint Piperita Rectified India commercial mint plant, and linseed oil.

In one aspect, the Rainforest Mint flavor is present from 0.5 to 1.5% of the flavor composition, for example, at 0.5%, or 0.6%, or 0.7%, or 0.8%, or 0.9%, or 1.0%, or 1.1%, or 1.2%, or 1.3%, or 1.4%, or 1.5%. In one aspect, the Rainforest Mint flavor is present at 1.21% of the flavor composition.

In one aspect, the anethol is present from 5 to 15% of the flavor composition, for example, at 5%, or 6%, or 7%, or 8%, or 9%, or 10%, or 11%, or 12%, or 13%, or 14%, or 15%. In one aspect, the anethol is present at 10.5% of the flavor composition.

In one aspect, the eucalyptus globulus is present from 1 to 4.0% of the flavor composition, for example, at 1%, or 1.5%, or 2%, or 2.5%, or 3%, or 3.5%, or 4.0%. In one aspect, eucalyptus globulus is present at 3.92% of the flavor composition.

In one aspect, the eugenol F is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the eugenol F is present at 0.45% of the flavor composition.

In one aspect, the glycyrrhizine ammoniacale @ 1% PG is present from 0.05 to 1.5% of the flavor composition, for example, at 0.05%, or 0.1%, or 0.15%, or 0.2%, or 0.25%, or 0.3%, or 0.35%, or 0.4%, or 0.45%, or 0.5%, or 0.55%, or 0.6%, or 0.65%, or 0.7%, or 0.75%, or 0.8%, or 0.85%, or 0.9%, or 0.95%, or 1%, or 1.5%. In one aspect, the glycyrrhizine ammoniacale @ 1% PG is present at 0.4% of the flavor composition.

In one aspect, the cooling compound known as Freeze 10 ethyl acetate is present from 0.5 to 10% of the flavor composition, for example, at 0.5%, or 0.6%, or 0.7%, or 0.8%, or 0.9%, or 1%, or 2%, or 3%, or 4%, or 5%, or 6%, or 7%, or 8%, or 9%, or 10%. In one aspect, the cooling compound known as Freeze 10 ethyl acetate is present at 1.31% of the flavor composition.

In one aspect, the menthol melted is present from 20 to 50% of the flavor composition, for example, at 20%, or 25%, or 30%, or 35%, or 40%, or 50%. In one aspect, the menthol melted is present at 43.85% of the flavor composition.

In one aspect, the menthol racemic is present from 2 to 8% of the flavor composition, for example, at 2%, or 3%, or 4%, or 5%, or 6%, or 7%, or 8%, of composition (a). In one aspect, the menthol racemic is present at 3.41% of the flavor composition.

In one aspect, the essential oil derived from a M. arvensis TPC commercial mint plant is present from 4 to 10% of the flavor composition, for example, at 4%, or 5%, or 6%, or 7%, or 8%, or 9%, or 10%. In one aspect, the essential oil derived from a M arvensis TPC commercial mint plant is present at 8.74% of the flavor composition.

In one aspect, the essential oil derived from a Euromint commercial mint plant is present from 0.5 to 2.5% of the flavor composition, for example, at 0.5%, or 1%, or 1.5%, or 2%, or 2.5%. In one aspect, the essential oil derived from a Euromint commercial mint plant is present at 0.55% of the flavor composition.

In one aspect, the essential oil derived from a Piperita ABS commercial mint plant @ 10% Mint Piperita Rectified India commercial mint plant is present from 0.01 to 0.1% of the flavor composition, for example, at 0.01%, or 0.02%, or 0.03%, or 0.04%, or 0.05%, or 0.06%, or 0.07%, or 0.08%, or 0.09%, or 0.10%. In one aspect, the essential oil derived from a Piperita ABS commercial mint plant @ 10% Mint Piperita Rectified India commercial mint plant is present at 0.02% of the flavor composition.

In one aspect, the essential oil derived from a 07-A3-5A hybrid mint plant is present from 0.5 to 2.5% of the flavor composition, for example, at 0.5%, or 1%, or 1.5%, or 2%, or 2.5%. In one aspect, the essential oil derived from a 07-A3-5A hybrid mint plant is present at 1% of the flavor composition.

In one aspect, the essential oil derived from a Mint Piperita Cascade commercial mint plant is present from 4 to 15% of the flavor composition, for example, at 4%, or 5%, or 6%, or 7%, or 8%, or 9%, or 10%, or 11%, or 12%, or 13%, or 14%, or 15%. In one aspect, the essential oil derived from a Mint Piperita Cascade commercial mint plant is present at 10.37% of the flavor composition.

In one aspect, the essential oil derived from a Mint Piperita Rectified India commercial mint plant is present from 4 to 15% of the flavor composition, for example, at 4%, or 5%, or 6%, or 7%, or 8%, or 9%, or 10%, or 11%, or 12%, or 13%, or 14%, or 15%. In one aspect, the essential oil derived from a Mint Piperita Rectified India commercial mint plant is present at 12.27% of the flavor composition.

In one aspect, the linseed oil is present from 0.5 to 2.5% of the flavor composition, for example, at 0.5%, or 1%, or 1.5%, or 2%, or 2.5%. In one aspect, the linseed oil is present at 2% of the flavor composition.

In one aspect, the flavor composition is described in Table 21.

In one aspect, the composition further comprises a perfume. Examples of perfumes include mixtures of natural and synthetic aromatic substances. Natural aromatic substances may include extracts of flowers (lily, lavender, rose, jasmine, neroli, ylang-ylang), stems, and leaves (geranium, patchouli, petitgrain), fruits (anise, coriander, caraway, juniper), fruit skins (bergamot, lemon, orange), roots (cumin, angelica, celery, cardamom, costus, iris, calmus), woods (pine, sandal, guaiac, cedar, rosewood), herbs and grasses (tarragon, lemon gras, sage, thyme), needles and branches (spruce, pine), resins and balms (galbanum, elemi, benzoin, myrrh, olibanum, opoponax). Animal-derived raw materials, e.g., civet and castoreum, may also be used. Typical synthetic aromatic compounds are products of the type of esters, ethers, aldehydes, ketones, alcohols, and hydrocarbons. Aromatic compounds of the type of esters include, e.g., benzyl acetate, phenoxxethyl isobutyrate, p-tert-butylcyclohexyl acetate, linalyl acetate, dimethylbenzylcarbinyl acetate, phenylethyl acetate, linalyl benzoate, benzyl formiate, ethylmethylphenyl glycinate, allylcyclohexyl propionate, styrallyl propionate, and benzyl salicylate. Ethers include, e.g., benzylethylethers, aldehydes include, e.g., the linear alkanals with 8-18 carbon atoms, citral, citronellal, citronellyl oxyacetaldehyde, cyclamenaldehyde, hydroxycitronellal, lilial, and bourgeonal, ketones include, e.g., a-isomethylionone and methylcedrylketone, alcohols include, e.g., anethol, citronellol, eugenol, isoeugenol, geraniol, linalool, phenylethyl alcohol and terpineol, and hydrocarbons primarily include terpenes and balms. In one aspect, the perfume is a mixture of various aromatic substances that, together, generate an appealing fragrance. Etheric oils with lower volatility, which are mostly used as aromatic components, are suitable as perfume oils, e.g., sage oil, camomile oil, clove oil, balm oil, mint oil, cinnamon leaf oil, lime blossom oil, juniper berry oil, vetiver oil, olibanum oil, galbanum oil, labdanum oil, and lavandin oil, bergamot oil, dihydromyrcenol, lilial, lyral, citronellol, phenylethyl alcohol, α-hexylcinnamic aldehyde, geraniol, benzyl acetone, cyclamen aldehyde, Boisambrene Forte, ambroxan, indol, hedione, sandelice, lemon oil, mandarin oil, orange oil, allylamyl glycolate, cyclovertal, lavandin oil, muscatel sage oil, 13-damascone, geranium oil, bourbon, cyclohexyl salicylate, Vertofix Coeur, Iso-E-Super, Fixolide NP, Evernyl, I raldein gamma, phenylacetic acid, geranyl acetate, benzyl acetate, rose oxide, romilate, irotyl, and floramate, alone or in mixtures, are used.

Additional perfumes include, e.g., peppermint oil, spearmint oil, anise oil, star aniseed oil, caraway oil, eucalyptus oil, fennel oil, lemon oil, wintergreen oil, clove oil, menthol, etc.

### Preparations Comprising the Flavor Compositions.

The flavor compositions described supra may be incorporated into preparations such as toothpastes, mouthwashes, perfumes, lotions, shaving creams, post shaving preparations, shampoos, antiperspirants, deodorants, and pharmaceutical products, among others.

Cosmetic preparations include, but are not limited to skin care, hair care, personal hygiene, sun protection, and oral and dental care products.

Accordingly, one aspect presented herein, provides a cosmetic composition comprising:
a. the flavor composition comprising:
   i. a combination selected from the group consisting of: l-carvone and d-limonene, wherein the weight ratio of the l-carvone to d-limonene in the combination is from 0.83:1 to 1.25:1; menthofuran and l-menthol, wherein the weight ratio of the menthofuran to l-menthol in the combination is from 0.87:1 to 1.31:1; l-menthol and l-menthyl acetate, wherein the weight ratio of the l-menthol to l-menthyl acetate in the combination is from 6.15:1 to 9.23:1; l-menthol and l-menthone, wherein the weight ratio of the l-menthol to l-menthone in the combination is from 1.66:1 to 2.50:1; l-menthol and 1-menthone, wherein the weight ratio of the l-menthol to l-menthone in the combination is from 0.98:1 to 1.47:1; l-menthol and l-menthone, wherein the weight ratio of the l-menthol to l-menthone in the combination is from 2.52:1 to 3.77:1; l-menthol and l-menthone, wherein the weight ratio of the l-menthol to l-menthone in the combination is from 1.71:1 to 2.61:1; 1-menthol and l-menthone, wherein the weight ratio of the l-menthol to l-menthone in the combination is from 1.73:1 to 2.60:1; l-menthol and menthofuran, wherein the weight ratio of the l-menthol to menthofuran in the combination is from 1.71:1 to 2.56:1; l-menthol and menthofuran, wherein the weight ratio of the l-menthol to menthofuran in the combination is from 1.70:1 to 2.55:1; l-menthone and l-menthol, wherein the weight ratio of the l-menthone to l-menthol in the combination is from 0.95:1 to 1.43:1; comprising l-menthone and l-menthol, wherein the weight ratio of the l-menthone to l-menthol in the combination is from 6.17:1 to 9.26:1; menthofuran and l-menthyl acetate, wherein the weight ratio of the menthofuran to l-menthyl acetate in the combination is from 1.92:1 to 2.89:1;
   ii. at least one cooling compound, and
b. a carrier suitable for cosmetic applications.

One aspect presented herein, provides a pharmaceutical composition comprising:
a. the flavor composition comprising:
   i. a combination selected from the group consisting of: l-carvone and d-limonene, wherein the weight ratio of the l-carvone to d-limonene in the combination is from 0.83:1 to 1.25:1; menthofuran and l-menthol, wherein the weight ratio of the menthofuran to l-menthol in the combination is from 0.87:1 to 1.31:1; l-menthol and l-menthyl acetate, wherein the weight ratio of the l-menthol to l-menthyl acetate in the combination is from 6.15:1 to 9.23:1; l-menthol and l-menthone, wherein the weight ratio of the l-menthol to l-menthone in the combination is from 1.66:1 to 2.50:1; l-menthol and 1-menthone, wherein the weight ratio of the l-menthol to l-menthone in the combination is from 0.98:1 to 1.47:1; l-menthol and l-menthone, wherein the weight ratio of the l-menthol to l-menthone in the combination is from 2.52:1 to 3.77:1; l-menthol and l-menthone, wherein the weight ratio of the l-menthol to l-menthone in the combination is from 1.71:1 to 2.61:1; 1-menthol and l-menthone, wherein the weight ratio of the l-menthol to 1-menthone in the combination is from 1.73:1 to 2.60:1; l-menthol and menthofuran, wherein the weight ratio of the l-menthol to menthofuran in the combination is from 1.71:1 to 2.56:1; l-menthol and menthofuran, wherein the weight ratio of the l-menthol to menthofuran in the combination is from 1.70:1 to 2.55:1; l-menthone and l-menthol, wherein the weight ratio of the l-menthone to l-menthol in the combination is from 0.95:1 to 1.43:1; comprising l-menthone and l-menthol, wherein the weight ratio of the l-menthone to l-menthol in the combination is from 6.17:1 to 9.26:1; menthofuran and l-menthyl acetate, wherein the weight ratio of the menthofuran to l-menthyl acetate in the combination is from 1.92:1 to 2.89:1;
   ii. at least one cooling compound, and
b. a carrier suitable for pharmaceutical applications.

One aspect presented herein, provides a food composition comprising:
a. the flavor composition comprising:
   i. a combination selected from the group consisting of: l-carvone and d-limonene, wherein the weight ratio of the l-carvone to d-limonene in the combination is from 0.83:1 to 1.25:1; menthofuran and l-menthol, wherein the weight ratio of the menthofuran to l-menthol in the combination is from 0.87:1 to 1.31:1; l-menthol and l-menthyl acetate, wherein the weight ratio of the l-menthol to l-menthyl acetate in the combination is from 6.15:1 to 9.23:1; l-menthol and l-menthone, wherein the weight ratio of the l-menthol to l-menthone in the combination is from 1.66:1 to 2.50:1; l-menthol and 1-menthone, wherein the weight ratio of the l-menthol to l-menthone in the combination is from 0.98:1 to 1.47:1; l-menthol and l-menthone, wherein the weight ratio of the l-menthol to l-menthone in the combination is from 2.52:1 to 3.77:1; l-menthol and l-menthone, wherein the weight ratio of the l-menthol to l-menthone in the combination is from 1.71:1 to 2.61:1; 1-menthol and l-menthone, wherein the weight ratio of the l-menthol to 1-menthone in the combination is from 1.73:1 to 2.60:1; l-menthol and menthofuran, wherein the weight ratio of the l-menthol to menthofuran in the combination is from 1.71:1 to 2.56:1; l-menthol and menthofuran, wherein the weight ratio of the l-menthol to menthofuran in the combination is from 1.70:1 to 2.55:1; 1-menthone and l-menthol, wherein the weight ratio of the l-menthone to l-menthol in the combination is from 0.95:1 to 1.43:1; comprising l-menthone and l-menthol, wherein the weight ratio of the 1-menthone to l-menthol in the combination is from 6.17:1 to 9.26:1; menthofuran and l-menthyl acetate, wherein the weight ratio of the menthofuran to l-menthyl acetate in the combination is from 1.92:1 to 2.89:1;
   ii. at least one cooling compound, and
b. a carrier suitable for food applications.

One aspect presented herein, provides a fragrance composition comprising:
a. the flavor composition comprising:
   i. a combination selected from the group consisting of: l-carvone and d-limonene, wherein the weight ratio of the l-carvone to d-limonene in the combination is from 0.83:1 to 1.25:1; menthofuran and l-menthol, wherein the weight ratio of the menthofuran to l-menthol in the combination is from 0.87:1 to 1.31:1; l-menthol and l-menthyl acetate, wherein the weight ratio of the l-menthol to l-menthyl acetate in the combination is from 6.15:1 to 9.23:1; l-menthol and l-menthone, wherein the weight ratio of the l-menthol to l-menthone in the combination is from 1.66:1 to 2.50:1; l-menthol and 1-menthone, wherein the weight ratio of the l-menthol to l-menthone in the combination is from 0.98:1 to 1.47:1; l-menthol and l-menthone, wherein the weight ratio of the l-menthol to l-menthone in the combination is from 2.52:1 to 3.77:1; l-menthol and l-menthone, wherein the weight ratio of the l-menthol to l-menthone in the combination is from 1.71:1 to 2.61:1; l-menthol and l-menthone, wherein the weight ratio of the l-menthol to 1-menthone in the combination is from 1.73:1 to 2.60:1; l-menthol and menthofuran, wherein the weight ratio of the l-menthol to menthofuran in the combination is from 1.71:1 to 2.56:1; l-menthol and menthofuran, wherein the weight ratio of the l-menthol to menthofuran in the combination is from 1.70:1 to 2.55:1; 1-menthone and l-menthol, wherein the weight ratio of the l-menthone to l-menthol in the combination is from 0.95:1 to 1.43:1; comprising l-menthone and l-menthol, wherein the weight ratio of the 1-menthone to l-menthol in the combination is from 6.17:1 to 9.26:1; menthofuran and l-menthyl acetate, wherein the weight ratio of the menthofuran to l-menthyl acetate in the combination is from 1.92:1 to 2.89:1;
   ii. at least one cooling compound, and
b. a carrier suitable for fragrance applications.

In one aspect, the combination is derived from a hybrid mint plant known as 13-S12-2. In one aspect, the combination is derived from a hybrid mint plant known as 13-39-9. In an alternate aspect, the combination is derived from a hybrid mint plant known as 13-A36-13. In an alternate aspect, the combination is derived from a hybrid mint plant known as 07-A3-11. In an alternate aspect, the combination is derived from a hybrid mint plant known as 09-6-2. In an alternate aspect, the combination is derived from a hybrid mint plant known as 11-A35-3. In an alternate aspect, the combination is derived from a hybrid mint plant known as 07-A3-5A. In an alternate aspect, the combination is derived from a hybrid mint plant known as 14-27-71. In an alternate aspect, the combination is derived from a hybrid mint plant known as 08-6-10. In an alternate aspect, the combination is derived from a hybrid mint plant known as 14-41-16. In an alternate aspect, the combination is derived from a hybrid mint plant known as 08-A20-3. In an alternate aspect, the combination is derived from a hybrid mint plant known as 14-27-89. In an alternate aspect, the combination is derived from a hybrid mint plant known as 05-19-1.

In one aspect, the combination further comprises at least one extract derived from a commercial mint plant.

One aspect presented herein, provides a cosmetic composition comprising:
a. the flavor composition comprising:
   i. a combination comprising l-carvone and d-limonene, wherein the weight ratio of the l-carvone to d-limonene in the combination is from 0.83:1 to 1.25:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-carvone and d-limonene,
      wherein the weight ratio of the l-carvone to d-limonene in the combination is from 0.83:1 to 1.25:1;
   ii. at least one cooling compound, and
b. a carrier suitable for pharmaceutical applications.

One aspect presented herein, provides a food composition comprising:
a. the flavor composition comprising:
   i. a combination comprising l-carvone and d-limonene,
   wherein the weight ratio of the l-carvone to d-limonene in the combination is from 0.83:1 to 1.25:1;
   ii. at least one cooling compound, and
b. a carrier suitable for food applications.

One aspect presented herein, provides a fragrance composition comprising:
a. the flavor composition comprising:
   i. a combination comprising l-carvone and d-limonene,
      wherein the weight ratio of the l-carvone to d-limonene in the combination is from 0.83:1 to 1.25:1;
   ii. at least one cooling compound, and
b. a carrier suitable for fragrance applications.

In one aspect, the combination is derived from a hybrid mint plant known as 13-S12-2.

One aspect presented herein, provides a cosmetic composition comprising:
a. the flavor composition comprising:
   i. a combination comprising menthofuran and l-menthol, wherein the weight ratio of the menthofuran to l-menthol in the combination is from 0.87:1 to 1.31:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising menthofuran and l-menthol,
      wherein the weight ratio of the menthofuran to l-menthol in the combination is from 0.87:1 to 1.31:1;
   ii. at least one cooling compound, and
b. a carrier suitable for pharmaceutical applications.

One aspect presented herein, provides a food composition comprising:
a. the flavor composition comprising:
   i. a combination comprising menthofuran and l-menthol,
      wherein the weight ratio of the menthofuran to l-menthol in the combination is from 0.87:1 to 1.31:1;
   ii. at least one cooling compound, and
b. a carrier suitable for food applications.

One aspect presented herein, provides a fragrance composition comprising:
a. the flavor composition comprising:
   i. a combination comprising menthofuran and l-menthol,
      wherein the weight ratio of the menthofuran to l-menthol in the combination is from 0.87:1 to 1.31:1;
   ii. at least one cooling compound, and
b. a carrier suitable for fragrance applications.

In one aspect, the combination is derived from a hybrid mint plant known as 13-39-9.

One aspect presented herein, provides a cosmetic composition comprising:
a. the flavor composition comprising:
   i. a combination comprising l-menthol and l-menthyl acetate,
      wherein the weight ratio of the l-menthol to l-menthyl acetate in the combination is from 6.15:1 to 9.23:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthyl acetate,
      wherein the weight ratio of the l-menthol to l-menthyl acetate in the combination is from 6.15:1 to 9.23:1;
   ii. at least one cooling compound, and
b. a carrier suitable for pharmaceutical applications.

One aspect presented herein, provides a food composition comprising:
a. the flavor composition comprising:
i. a combination comprising l-menthol and l-menthyl acetate, wherein the weight ratio of the l-menthol to l-menthyl acetate in the combination is from 6.15:1 to 9.23:1;
   ii. at least one cooling compound, and
b. a carrier suitable for food applications.

One aspect presented herein, provides a fragrance composition comprising:
a. the flavor composition comprising:
   i. a combination comprising l-menthol and l-menthyl acetate,
      wherein the weight ratio of the l-menthol to l-menthyl acetate in the combination is from 6.15:1 to 9.23:1;
   ii. at least one cooling compound, and
b. a carrier suitable for fragrance applications.

In one aspect, the combination is derived from a hybrid mint plant known as 13-A36-13.

One aspect presented herein, provides a cosmetic composition comprising:
a. the flavor composition comprising:
   i. a combination comprising l-menthol and l-menthone,
      wherein the weight ratio of the l-menthol to l-menthone in the combination is from 1.66:1 to 2.50:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthone,
      wherein the weight ratio of the l-menthol to l-menthone in the combination is from 1.66:1 to 2.50:1;
   ii. at least one cooling compound, and
b. a carrier suitable for pharmaceutical applications.

One aspect presented herein, provides a food composition comprising:
a. the flavor composition comprising:
   i. a combination comprising l-menthol and l-menthone,
      wherein the weight ratio of the l-menthol to l-menthone in the combination is from 1.66:1 to 2.50:1;
   ii. at least one cooling compound, and
b. a carrier suitable for food applications.

One aspect presented herein, provides a fragrance composition comprising:
a. the flavor composition comprising:
   i. a combination comprising l-menthol and l-menthone,
      wherein the weight ratio of the l-menthol to l-menthone in the combination is from 1.66:1 to 2.50:1;
   ii. at least one cooling compound, and
b. a carrier suitable for fragrance applications.

In one aspect, the combination is derived from a hybrid mint plant known as 07-A3-11.

One aspect presented herein, provides a cosmetic composition comprising:
a. the flavor composition comprising:
   i. a combination comprising l-menthol and l-menthone,
      wherein the weight ratio of the l-menthol to l-menthone in the combination is from 0.98:1 to 1.47:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthone, wherein the weight ratio of the l-menthol to l-menthone in the combination is from 0.98:1 to 1.47:1;
   ii. at least one cooling compound, and
b. a carrier suitable for pharmaceutical applications.

One aspect presented herein, provides a food composition comprising:
a. the flavor composition comprising:
   i. a combination comprising l-menthol and l-menthone,
      wherein the weight ratio of the l-menthol to l-menthone in the combination is from 0.98:1 to 1.47:1;
   ii. at least one cooling compound, and
b. a carrier suitable for food applications.

One aspect presented herein, provides a fragrance composition comprising:
a. the flavor composition comprising:
   i. a combination comprising l-menthol and l-menthone,
      wherein the weight ratio of the l-menthol to l-menthone in the combination is from 0.98:1 to 1.47:1;
   ii. at least one cooling compound, and
b. a carrier suitable for fragrance applications.

In one aspect, the combination is derived from a hybrid mint plant known as 09-6-2.

One aspect presented herein, provides a cosmetic composition comprising:
a. the flavor composition comprising:
   i. a combination comprising l-menthol and l-menthone,
      wherein the weight ratio of the l-menthol to l-menthone in the combination is from 2.52:1 to 3.77:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthone,
      wherein the weight ratio of the l-menthol to l-menthone in the combination is from 2.52:1 to 3.77:1;
   ii. at least one cooling compound, and
b. a carrier suitable for pharmaceutical applications.

One aspect presented herein, provides a food composition comprising:
a. the flavor composition comprising:
   i. a combination comprising l-menthol and l-menthone,
      wherein the weight ratio of the l-menthol to l-menthone in the combination is from 2.52:1 to 3.77:1;
   ii. at least one cooling compound, and
b. a carrier suitable for food applications.

One aspect presented herein, provides a fragrance composition comprising:
a. the flavor composition comprising:
   i. a combination comprising l-menthol and l-menthone,
      wherein the weight ratio of the l-menthol to l-menthone in the combination is from 2.52:1 to 3.77:1;
   ii. at least one cooling compound, and
b. a carrier suitable for fragrance applications.

In one aspect, the combination is derived from a hybrid mint plant known as 11-A35-3.

One aspect presented herein, provides a cosmetic composition comprising:
a. the flavor composition comprising:
   i. a combination comprising l-menthol and l-menthone,
      wherein the weight ratio of the l-menthol to l-menthone in the combination is from 1.71:1 to 2.61:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthone,
      wherein the weight ratio of the l-menthol to l-menthone in the combination is from 1.71:1 to 2.61:1;
   ii. at least one cooling compound, and
b. a carrier suitable for pharmaceutical applications.

One aspect presented herein, provides a food composition comprising:
a. the flavor composition comprising:
   i. a combination comprising l-menthol and l-menthone,
      wherein the weight ratio of the l-menthol to l-menthone in the combination is from 1.71:1 to 2.61:1;
   ii. at least one cooling compound, and
b. a carrier suitable for food applications.

One aspect presented herein, provides a fragrance composition comprising:
a. the flavor composition comprising:
   i. a combination comprising l-menthol and l-menthone,
      wherein the weight ratio of the l-menthol to l-menthone in the combination is from 1.71:1 to 2.61:1;
   ii. at least one cooling compound, and
b. a carrier suitable for fragrance applications.

In one aspect, the combination is derived from a hybrid mint plant known as 07-A3-5A.

One aspect presented herein, provides a cosmetic composition comprising:
a. the flavor composition comprising:
   i. a combination comprising l-menthol and l-menthone,
      wherein the weight ratio of the l-menthol to l-menthone in the combination is from 1.73:1 to 2.60:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and l-menthone,
      wherein the weight ratio of the l-menthol to l-menthone in the combination is from 1.73:1 to 2.60:1;
   ii. at least one cooling compound, and
b. a carrier suitable for pharmaceutical applications.

One aspect presented herein, provides a food composition comprising:
a. the flavor composition comprising:
   i. a combination comprising l-menthol and l-menthone,
      wherein the weight ratio of the l-menthol to l-menthone in the combination is from 1.73:1 to 2.60:1;
   ii. at least one cooling compound, and
b. a carrier suitable for food applications.

One aspect presented herein, provides a fragrance composition comprising:
a. the flavor composition comprising:
   i. a combination comprising l-menthol and l-menthone,
      wherein the weight ratio of the l-menthol to l-menthone in the combination is from 1.73:1 to 2.60:1;
   ii. at least one cooling compound, and
b. a carrier suitable for fragrance applications.

In one aspect, the combination is derived from a hybrid mint plant known as 14-27-71.

One aspect presented herein, provides a cosmetic composition comprising:
a. the flavor composition comprising:
   i. a combination comprising l-menthol and menthofuran,
      wherein the weight ratio of the l-menthol to menthofuran in the combination is from 1.71:1 to 2.56:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and menthofuran,
      wherein the weight ratio of the l-menthol to menthofuran in the combination is from 1.71:1 to 2.56:1;
   ii. at least one cooling compound, and
b. a carrier suitable for pharmaceutical applications.

One aspect presented herein, provides a food composition comprising:
a. the flavor composition comprising:
   i. a combination comprising l-menthol and menthofuran,
      wherein the weight ratio of the l-menthol to menthofuran in the combination is from 1.71:1 to 2.56:1;
   ii. at least one cooling compound, and
b. a carrier suitable for food applications.

One aspect presented herein, provides a fragrance composition comprising:
a. the flavor composition comprising:
   i. a combination comprising l-menthol and menthofuran,
   wherein the weight ratio of the l-menthol to menthofuran in the combination is from 1.71:1 to 2.56:1;
   ii. at least one cooling compound, and
b. a carrier suitable for fragrance applications.

In one aspect, the combination is derived from a hybrid mint plant known as 08-6-10.

One aspect presented herein, provides a cosmetic composition comprising:
a. the flavor composition comprising:
   i. a combination comprising l-menthol and menthofuran,
      wherein the weight ratio of the l-menthol to menthofuran in the combination is from 1.70:1 to 2.55:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthol and menthofuran,
      wherein the weight ratio of the l-menthol to menthofuran in the combination is from 1.70:1 to 2.55:1;
   ii. at least one cooling compound, and
b. a carrier suitable for pharmaceutical applications.

One aspect presented herein, provides a food composition comprising:
a. the flavor composition comprising:
   i. a combination comprising l-menthol and menthofuran,
      wherein the weight ratio of the l-menthol to menthofuran in the combination is from 1.70:1 to 2.55:1;
   ii. at least one cooling compound, and
b. a carrier suitable for food applications.

One aspect presented herein, provides a fragrance composition comprising:
a. the flavor composition comprising:
   i. a combination comprising l-menthol and menthofuran,
      wherein the weight ratio of the l-menthol to menthofuran in the combination is from 1.70:1 to 2.55:1;
   ii. at least one cooling compound, and
b. a carrier suitable for fragrance applications.

In one aspect, the combination is derived from a hybrid mint plant known as 14-41-16.

One aspect presented herein, provides a cosmetic composition comprising:
a. the flavor composition comprising:
   i. a combination comprising l-menthone and l-menthol,
      wherein the weight ratio of the l-menthone to l-menthol in the combination is from 0.95:1 to 1.43:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthone and l-menthol,
      wherein the weight ratio of the l-menthone to l-menthol in the combination is from 0.95:1 to 1.43:1;
   ii. at least one cooling compound, and
b. a carrier suitable for pharmaceutical applications.

One aspect presented herein, provides a food composition comprising:
a. the flavor composition comprising:
   i. a combination comprising l-menthone and l-menthol,
      wherein the weight ratio of the l-menthone to l-menthol in the combination is from 0.95:1 to 1.43:1;
   ii. at least one cooling compound, and
b. a carrier suitable for food applications.

One aspect presented herein, provides a fragrance composition comprising:
a. the flavor composition comprising:
   i. a combination comprising l-menthone and l-menthol,
      wherein the weight ratio of the l-menthone to l-menthol in the combination is from 0.95:1 to 1.43:1;
   ii. at least one cooling compound, and
b. a carrier suitable for fragrance applications.

In one aspect, the combination is derived from a hybrid mint plant known as 08-A20-3.

One aspect presented herein, provides a cosmetic composition comprising:
a. the flavor composition comprising:
   i. a combination comprising l-menthone and l-menthol,
      wherein the weight ratio of the l-menthone to l-menthol in the combination is from 6.17:1 to 9.26:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising l-menthone and l-menthol,
      wherein the weight ratio of the l-menthone to l-menthol in the combination is from 6.17:1 to 9.26:1;
   ii. at least one cooling compound, and
b. a carrier suitable for pharmaceutical applications.

One aspect presented herein, provides a food composition comprising:
a. the flavor composition comprising:
   i. a combination comprising l-menthone and l-menthol,
      wherein the weight ratio of the l-menthone to l-menthol in the combination is from 6.17:1 to 9.26:1;
   ii. at least one cooling compound, and
b. a carrier suitable for food applications.

One aspect presented herein, provides a fragrance composition comprising:
a. the flavor composition comprising:
   i. a combination comprising l-menthone and l-menthol,
      wherein the weight ratio of the l-menthone to l-menthol in the combination is from 6.17:1 to 9.26:1;
   ii. at least one cooling compound, and
b. a carrier suitable for fragrance applications.

In one aspect, the combination is derived from a hybrid mint plant known as 14-27-89.

One aspect presented herein, provides a cosmetic composition comprising:
a. the flavor composition comprising:
   i. a combination comprising menthofuran and l-menthyl acetate,
      wherein the weight ratio of the menthofuran to l-menthyl acetate in the combination is from 1.92:1 to 2.89:1;
   ii. at least one cooling compound, and
b. a carrier suitable for cosmetic applications.

In one aspect, the cosmetic preparation is an oral care composition.

One aspect presented herein, provides a pharmaceutical composition comprising:
a. the flavor composition comprising:
   i. a combination comprising menthofuran and l-menthyl acetate,
      wherein the weight ratio of the menthofuran to l-menthyl acetate in the combination is from 1.92:1 to 2.89:1;
   ii. at least one cooling compound, and
b. a carrier suitable for pharmaceutical applications.

One aspect presented herein, provides a food composition comprising:
a. the flavor composition comprising:
   i. a combination comprising menthofuran and l-menthyl acetate,
      wherein the weight ratio of the menthofuran to l-menthyl acetate in the combination is from 1.92:1 to 2.89:1;
   ii. at least one cooling compound, and
b. a carrier suitable for food applications.

One aspect presented herein, provides a fragrance composition comprising:
a. the flavor composition comprising:
   i. a combination comprising menthofuran and l-menthyl acetate,
      wherein the weight ratio of the menthofuran to l-menthyl acetate in the combination is from 1.92:1 to 2.89:1;
   ii. at least one cooling compound, and
b. a carrier suitable for fragrance applications.

In one aspect, the combination is derived from a hybrid mint plant known as 05-19-1.

In one aspect, the at least one cooling compound may be present in the preparation at an amount from 0.005 to 0.4%. In one aspect, the at least one cooling compound is present at 0.4%, alternatively, at 0.3%, alternatively, at 0.2%, alternatively, at 0.1%, alternatively, at 0.05%, alternatively, at 0.04%, alternatively, at 0.03%, alternatively, at 0.02%, alternatively, at 0.01%, alternatively, at 0.009%, alternatively, at 0.008%, alternatively, at 0.007%, alternatively, at 0.006%, alternatively, at 0.005%.

In one aspect, the cosmetic preparation is an oral care composition, such as, for example, a toothpaste.

Toothpastes are generally gel or pasty preparations of water, thickeners, humectants, abrasives, surfactants, sweeteners, scents, deodorants, as well as agents against oral and dental diseases. Toothpastes according to certain aspects presented herein may include all standard abrasives, e.g., chalk, dicalcium phosphate, unsoluble sodium metaphosphate, aluminium silicates, calcium pyrophosphate, fine synthetic resins, silicic acids, aluminium oxide, and aluminium oxide trihydrate.

Ingredients suitable to formulate oral and/or dental care products according to certain aspects presented herein are disclosed in International Patent Application Publication No. WO2014090293.

In one aspect, the oral care preparation comprises anethol, carvone gauche, the cooling compound known as Freeze 10 ethyl acetate, menthol melted, menthol racemic, terpeneless essential oil derived from a M arvensis mint plant, an essential oil derived from a 07-A3-5A hybrid mint plant, an essential oil derived from a Mint Piperita Rectified India commercial mint plant, an essential oil derived from a Spicata Native commercial mint plant, an essential oil derived from a Spicata US commercial mint plant, and linseed oil.

In one aspect, the anethol is present in the oral care preparation in an amount from 5 to 15%, for example, at 5%, or 6%, or 7%, or 8%, or 9%, or 10%, or 11%, or 12%, or 13%, or 14%, or 15%. In one aspect, the amount of the anethol in the oral care preparation is 9.3%.

In one aspect, the carvone gauche is present in the oral care preparation in an amount from 5 to 15%, for example, at 5%, or 6%, or 7%, or 8%, or 9%, or 10%, or 11%, or 12%, or 13%, or 14%, or 15%. In one aspect, the amount of carvone gauche in the oral care preparation is 8.8%.

In one aspect, the cooling compound known as Freeze 10 ethyl acetate is present in the oral care preparation in an amount from 0.5 to 10%, for example, at 0.5%, or 0.6%, or 0.7%, or 0.8%, or 0.9%, or 1%, or 2%, or 3%, or 4%, or 5%, or 6%, or 7%, or 8%, or 9%, or 10%. In one aspect, the amount of the cooling compound known as Freeze 10 ethyl acetate in the oral care preparation is 0.9%.

In one aspect, the menthol melted is present from 20 to 40% of the oral care composition, for example, at 20%, or 25%, or 30%, or 35%, or 40%. In one aspect, the menthol melted is present at 27.3% of the oral care composition.

In one aspect, the menthol racemic is present in the oral care preparation in an amount from 4 to 7%, for example, at 4%, or 5%, or 6%, or 7%. In one aspect, the amount of the menthol racemic in the oral care preparation is 5.5%.

In one aspect, the terpeneless essential oil derived from a M arvensis mint plant is present in the oral care preparation in an amount from 4 to 7%, for example, at 4%, or 5%, or 6%, or 7%. In one aspect, the amount of the terpeneless essential oil derived from a M arvensis mint plant in the oral care preparation is 6.5%.

In one aspect, the essential oil derived from a 07-A3-5A hybrid mint plant is present in the oral care preparation in an amount from 1 to 2.5%, for example, at 1%, or 1.5%, or 2%, or 2.5%. In one aspect, the amount of the essential oil derived from a 07-A3-5A hybrid mint plant in the oral care preparation is 2.5%.

In one aspect, the essential oil derived from a Mint Piperita Rectified India commercial mint plant is present in the oral care preparation in an amount from 4 to 7%, for example, at 4%, or 5%, or 6%, or 7%. In one aspect, the amount of the essential oil derived from a Mint Piperita Rectified India commercial mint plant in the oral care preparation is 6.8%.

In one aspect, the essential oil derived from a Spicata Native commercial mint plant is present in the oral care preparation in an amount from 8 to 15%, for example, at 8%, or 9%, or 10%, or 11%, or 12%, or 13%, or 14%, or 15%. In one aspect, the amount of the essential oil derived from a Spicata Native commercial mint plant in the oral care preparation is 9.1%.

In one aspect, the essential oil derived from a Spicata US commercial mint plant is present in the oral care preparation in an amount from 8 to 15%, for example, at 8%, or 9%, or 10%, or 11%, or 12%, or 13%, or 14%, or 15%. In one aspect, the amount of the essential oil derived from a Spicata US commercial mint plant in the oral care preparation is 10.4%.

In one aspect, the linseed oil is present in the oral care preparation in an amount from 8 to 15%, for example, at 8%, or 9%, or 10%, or 11%, or 12%, or 13%, or 14%, or 15%. In one aspect, the amount of the linseed oil in the oral care preparation is 12.9%.

In one aspect, the oral care preparation is an oral care composition as described in Table 19.

Pharmaceutical preparations include, but are not limited to lozenges, cough drops, cold syrups, cold ointments, and cold sprays.

The cosmetic or pharmaceutical carriers may be selected from the group of water, alcohols with 2-6 carbon atoms, polyols with 1-10 carbon atoms and 2-4 hydroxyl groups, as well as oil particles. Particularly preferred in addition to water are ethanol, isopropyl alcohol, ethylene glycol, propylene glycol, glycerin, trimethylol propane, pentaerythrytol, and esters of linear or cross-linked, saturated, in particular unsaturated fatty acids with 6-22 and alternatively 8-18 carbon atoms with alcohols with 1-6 carbon atoms.

The cosmetic and/or pharmaceutical preparations according to certain aspects presented herein may contain additional typical excipients and additives, e.g. mild surfactants, oil particles, emulsifiers, pearlised waxes, consistency builders, thickeners, superfattening agents, stabilisiers, polymers, silicone compounds, fats, waxes, lecithins, phospholipids, UV protection factors, humectants, biogenic agents, antioxidants, deodorants, antiperspirants, antidandruff agents, film formers, swelling agents, insect repellants, self-tanning lotions, tyrosine inhibitors (depigmentation agents), hydrotropes, solubilisers, preservatives, perfume oils, and colourants, etc. Examples of additional excipients suitable for use in the aspects presented herein are disclosed in International Patent Application Publication No. WO2014090293.

In one aspect, the cosmetic preparation, or the pharmaceutical preparation further comprises a colorant. Examples of colorants suitable for use in the aspects presented herein are disclosed in International Patent Application Publication No. WO2014090293.

Examples of fragrance preparations include air care products, such as air fresheners, and the like.

### The Plant Extract.

In one aspect presented herein, the combination used in the flavor composition presented herein is derived from an extract of a hybrid mint plant. In an alternate aspect, the combination is derived from an extract of a hybrid mint plant and an extract of a commercial mint plant. In certain embodiments, the extract comprises an essential oil. As used herein, the term “essential oil” refers to a hydrophobic liquid containing volatile aroma compounds.

In one aspect, the essential oil derived from a hybrid mint plant differs from an essential oil derived from a commercially grown mint plant. For example, the relative ratios of the component aroma compounds in the essential oil derived from a hybrid mint plant may differ from the relative ratios of the component aroma compounds an essential oil derived from a commercially grown mint plant.

Without intending to be limited to any particular theory, the at least one extract derived from a hybrid mint plant, is intended to provide desired cooling properties to the flavor compositions according to certain aspects presented herein. Desired cooling properties may be imparted by the particular aromatic compounds, and/or the relative amounts of the particular aromatic compounds present in the at least one extract derived from a hybrid mint plant. Moreover, desired cooling properties may be imparted by blending several extracts, thereby giving rise to a specific mixture of aromatic compounds and/or different relative amounts of the particular aromatic compounds.

In one aspect, concentration of carvone in the essential oil derived from a hybrid mint plant is lower than the concentration of carvone in an essential oil derived from commercially grown mint plants, and the concentration of d-limonene in the essential oil derived from a hybrid mint plant is higher than the concentration of d-limonene in an essential oil derived from a commercially grown mint plant.

In one aspect, the concentration of carvone in the essential oil derived from a hybrid mint plant is from 5% to 90% lower. Alternatively, the concentration of carvone in the essential oil derived from a hybrid mint plant is from 10% to 90% lower. Alternatively, the concentration of carvone in the essential oil derived from a hybrid mint plant is from 10% to 80% lower. Alternatively, the concentration of carvone in the essential oil derived from a hybrid mint plant is from 10% to 70% lower. Alternatively, the concentration of carvone in the essential oil derived from a hybrid mint plant is from 10% to 60% lower. Alternatively, the concentration of carvone in the essential oil derived from a hybrid mint plant is from 10% to 50% lower. In one aspect, the concentration of carvone in the essential oil derived from a hybrid mint plant is 90% lower. Alternatively, the concentration of carvone in the essential oil derived from a hybrid mint plant is 80% lower. Alternatively, the concentration of carvone in the essential oil derived from a hybrid mint plant is 70% lower. Alternatively, the concentration of carvone in the essential oil derived from a hybrid mint plant is 60% lower. Alternatively, the concentration of carvone in the essential oil derived from a hybrid mint plant is 50% lower.

In one aspect, the concentration of d-limonene in the essential oil derived from a hybrid mint plant is from 100% to 300% higher. Alternatively, the concentration of d-limonene in the essential oil derived from a hybrid mint plant is from 100% to 200% higher. Alternatively, the concentration of d-limonene in the essential oil derived from a hybrid mint plant is from 100% to 150% higher. In one aspect, the concentration of d-limonene in the essential oil derived from a hybrid mint plant is 150% higher. Alternatively, the concentration of d-limonene in the essential oil derived from a hybrid mint plant is 200% higher. Alternatively, the concentration of d-limonene in the essential oil derived from a hybrid mint plant is 250% higher. Alternatively, the concentration of d-limonene in the essential oil derived from a hybrid mint plant is 300% higher.

In one aspect, the concentration of l-carvone in the essential oil derived from a hybrid mint plant is from 30 to 70%, determined by calculation of the relative peak areas of a gas chromatograph of the at least one essential oil. In one aspect, the concentration of 1-carvone in the essential oil derived from a hybrid mint plant is from 30 to 60%, alternatively, from 30 to 50%, alternatively, from 30 to 40%, alternatively, from 30 to 35%, or alternatively, from 30 to 32%. In one aspect, the concentration of carvone in the essential oil derived from a hybrid mint plant is 31.5%, determined by calculation of the relative peak areas of a gas chromatograph of the at least one essential oil.

In one aspect, the concentration of d-limonene in the essential oil derived from a hybrid mint plant is from 10 to 40%, determined by calculation of the relative peak areas of a gas chromatograph of the at least one essential oil. In one aspect, the concentration of d-limonene in the essential oil derived from a hybrid mint plant is from 10 to 35%, alternatively, from 10 to 30%, alternatively, from 10 to 25%, alternatively, from 10 to 20%, or alternatively, from 10 to 15%. In one aspect, the concentration of d-limonene in the essential oil derived from a hybrid mint plant is 30.3%, determined by calculation of the relative peak areas of a gas chromatograph of the at least one essential oil.

In one aspect, the concentration of l-menthone and l-menthol in the essential oil derived from a hybrid mint plant is lower than the concentration of l-menthone and l-menthol in an essential oil derived from a commercially grown mint plant.

In one aspect, the concentration of l-menthone in the essential oil derived from a hybrid mint plant is from 1% to 50% lower. Alternatively, the concentration of l-menthone in the essential oil derived from a hybrid mint plant is from 1% to 45% lower. Alternatively, the concentration of l-menthone in the essential oil derived from a hybrid mint plant is from 1% to 40% lower. Alternatively, the concentration of l-menthone in the essential oil derived from a hybrid mint plant is from 1% to 35% lower. Alternatively, the concentration of l-menthone in the essential oil derived from a hybrid mint plant is from 1% to 30% lower. Alternatively, the concentration of l-menthone in the essential oil derived from a hybrid mint plant is from 1% to 25% lower. Alternatively, the concentration of l-menthone in the essential oil derived from a hybrid mint plant is from 1% to 20% lower. Alternatively, the concentration of l-menthone in the essential oil derived from a hybrid mint plant is from 1% to 15% lower. Alternatively, the concentration of l-menthone in the essential oil derived from a hybrid mint plant is from 1% to 10% lower. Alternatively, the concentration of l-menthone in the essential oil derived from a hybrid mint plant is from 1% to 5% lower. In one aspect, the concentration of l-menthone in the essential oil derived from a hybrid mint plant is 50% lower. Alternatively, the concentration of l-menthone in the essential oil derived from a hybrid mint plant is 45% lower. Alternatively, the concentration of l-menthone in the essential oil derived from a hybrid mint plant is 40% lower. Alternatively, the concentration of l-menthone in the essential oil derived from a hybrid mint plant is 35% lower. Alternatively, the concentration of l-menthone in the essential oil derived from a hybrid mint plant is 30% lower. Alternatively, the concentration of l-menthone in the essential oil derived from a hybrid mint plant is 25% lower. Alternatively, the concentration of l-menthone in the essential oil derived from a hybrid mint plant is 20% lower. Alternatively, the concentration of l-menthone in the essential oil derived from a hybrid mint plant is 15% lower. Alternatively, the concentration of l-menthone in the essential oil derived from a hybrid mint plant is 10% lower. Alternatively, the concentration of l-menthone in the essential oil derived from a hybrid mint plant is 5% lower.

In one aspect, the concentration of l-menthol in the essential oil derived from a hybrid mint plant is from 1% to 50% lower. Alternatively, the concentration of l-menthol in the essential oil derived from a hybrid mint plant is from 1% to 45% lower. Alternatively, the concentration of l-menthol in the essential oil derived from a hybrid mint plant is from 1% to 40% lower. Alternatively, the concentration of l-menthol in the essential oil derived from a hybrid mint plant is from 1% to 35% lower. Alternatively, the concentration of l-menthol in the essential oil derived from a hybrid mint plant is from 1% to 30% lower. Alternatively, the concentration of l-menthol in the essential oil derived from a hybrid mint plant is from 1% to 25% lower. Alternatively, the concentration of 1-menthol in the essential oil derived from a hybrid mint plant is from 1% to 20% lower. Alternatively, the concentration of l-menthol in the essential oil derived from a hybrid mint plant is from 1% to 15% lower. Alternatively, the concentration of l-menthol in the essential oil derived from a hybrid mint plant is from 1% to 10% lower. Alternatively, the concentration of l-menthol in the essential oil derived from a hybrid mint plant is from 1% to 5% lower. In one aspect, the concentration of l-menthol in the essential oil derived from a hybrid mint plant is 50% lower. Alternatively, the concentration of l-menthol in the essential oil derived from a hybrid mint plant is 45% lower. Alternatively, the concentration of l-menthol in the essential oil derived from a hybrid mint plant is 40% lower. Alternatively, the concentration of l-menthol in the essential oil derived from a hybrid mint plant is 35% lower. Alternatively, the concentration of l-menthol in the essential oil derived from a hybrid mint plant is 30% lower. Alternatively, the concentration of l-menthol in the essential oil derived from a hybrid mint plant is 25% lower. Alternatively, the concentration of l-menthol in the essential oil derived from a hybrid mint plant is 20% lower. Alternatively, the concentration of l-menthol in the essential oil derived from a hybrid mint plant is 15% lower. Alternatively, the concentration of l-menthol in the essential oil derived from a hybrid mint plant is 10% lower. Alternatively, the concentration of l-menthol in the essential oil derived from a hybrid mint plant is 5% lower.

In one aspect, the concentration of l-menthone in the essential oil derived from a hybrid mint plant is from 2 to 70%, determined by calculation of the relative peak areas of a gas chromatograph of the at least one essential oil. In one aspect, the concentration of carvone in the essential oil derived from a hybrid mint plant is from 2 to 60%, alternatively, from 2 to 50%, alternatively, from 2 to 40%, alternatively, from 2 to 35%, alternatively, from 2 to 30%, alternatively, from 2 to 20%, alternatively, from 2 to 10%, alternatively, from 2 to 5%.

In one aspect, the concentration of l-menthol in the essential oil derived from a hybrid mint plant is from 10 to 70%, determined by calculation of the relative peak areas of a gas chromatograph of the at least one essential oil. In one aspect, the concentration of l-menthol in the essential oil derived from a hybrid mint plant is from 10 to 60%, alternatively, from 10 to 50%, alternatively, from 10 to 40%, alternatively, from 10 to 35%, alternatively, from 10 to 30%, alternatively, from 10 to 20%, alternatively, from 10 to 15%.

In one aspect, the concentration of l-menthone in the essential oil derived from a hybrid mint plant is greater than the concentration of l-menthone in an essential oil derived from a commercially grown mint plant. In one aspect, the concentration of menthofuran in the essential oil derived from a hybrid mint plant is also greater than the concentration of menthofuran in an essential oil derived from a commercially grown mint plant.

In one aspect, the concentration of l-menthone in the essential oil derived from a hybrid mint plant is from 10% to 300% greater. Alternatively, the concentration of 1-menthone in the essential oil derived from a hybrid mint plant is from 10% to 250% greater. Alternatively, the concentration of l-menthone in the essential oil derived from a hybrid mint plant is from 10% to 200% greater. Alternatively, the concentration of l-menthone in the essential oil derived from a hybrid mint plant is from 10% to 150% greater. Alternatively, the concentration of l-menthone in the essential oil derived from a hybrid mint plant is from 10% to 100% greater. Alternatively, the concentration of l-menthone in the essential oil derived from a hybrid mint plant is from 10% to 50% greater. Alternatively, the concentration of l-menthone in the essential oil derived from a hybrid mint plant is from 10% to 40% greater. Alternatively, the concentration of l-menthone in the essential oil derived from a hybrid mint plant is from 10% to 30% greater. Alternatively, the concentration of l-menthone in the essential oil derived from a hybrid mint plant is from 10% to 20% greater.

In one aspect, the concentration of l-menthone in the essential oil derived from a hybrid mint plant is from 20 to 70%, determined by calculation of the relative peak areas of a gas chromatograph of the at least one essential oil. In one aspect, the concentration of carvone in the essential oil derived from a hybrid mint plant is from 20 to 60%, alternatively, from 20 to 50%, alternatively, from 20 to 40%, alternatively, from 20 to 35%, alternatively, from 20 to 30%, alternatively, from 2 to 25%.

In one aspect, the concentration of menthofuran is in the essential oil derived from a hybrid mint plant is from 10% to 500% greater. Alternatively, the concentration of menthofuran in the essential oil derived from a hybrid mint plant is from 10% to 400% greater. Alternatively, the concentration of menthofuran in the essential oil derived from a hybrid mint plant is from 10% to 300% greater. Alternatively, the concentration of menthofuran in the essential oil derived from a hybrid mint plant is from 10% to 200% greater. Alternatively, the concentration of menthofuran in the essential oil derived from a hybrid mint plant is from 10% to 100% greater. Alternatively, the concentration of menthofuran in the essential oil derived from a hybrid mint plant is from 10% to 50% greater.

In one aspect, the concentration of menthofuran in the essential oil derived from a hybrid mint plant is from 0.3 to 50%, determined by calculation of the relative peak areas of a gas chromatograph of the at least one essential oil. In one aspect, the concentration of menthofuran in the essential oil derived from a hybrid mint plant is from 1 to 50%, alternatively, from 10 to 50%, alternatively, from 10 to 40%, alternatively, from 10 to 35%, alternatively, from 10 to 30%, alternatively, from 10 to 20%, alternatively, from 10 to 15%.

In one aspect, the essential oil derived from a hybrid mint plant comprises between 0.5 and 1.5% a-pinene, determined by calculation of the relative peak areas of a gas chromatograph of the at least one essential oil. In one aspect, the essential oil derived from a hybrid mint plant comprises between 0.8 and 1.5% a-pinene. In one aspect, the essential oil derived from a hybrid mint plant comprises between 0.8 and 1% a-pinene.

In one aspect, the essential oil derived from a hybrid mint plant comprises between 0.5 and 1.5% β-pinene, determined by calculation of the relative peak areas of a gas chromatograph of the at least one essential oil. In one aspect, the essential oil derived from a hybrid mint plant comprises between 1 and 1.5% β-pinene. In one aspect, the essential oil derived from a hybrid mint plant comprises between 1 and 1.1% β-pinene.

In one aspect, the essential oil derived from a hybrid mint plant comprises between 1 and 4% l-limonene, determined by calculation of the relative peak areas of a gas chromatograph of the at least one essential oil. In one aspect, the essential oil derived from a hybrid mint plant comprises between 1 and 3.5% l-limonene. In one aspect, the essential oil derived from a hybrid mint plant comprises between 1 and 3% l-limonene. In one aspect, the essential oil derived from a hybrid mint plant comprises between 1 and 2.5% l-limonene. In one aspect, the essential oil derived from a hybrid mint plant comprises between 1 and 1% l-limonene. In one aspect, the essential oil derived from a hybrid mint plant comprises between 1 and 1.5% l-limonene.

In one aspect, the essential oil derived from a hybrid mint plant comprises between 1 and 7% 1,8-cineole, determined by calculation of the relative peak areas of a gas chromatograph of the at least one essential oil. In one aspect, the essential oil derived from a hybrid mint plant comprises between 1 and 6% 1,8-cineole. In one aspect, the essential oil derived from a hybrid mint plant comprises between 1 and 5% 1,8-cineole. In one aspect, the essential oil derived from a hybrid mint plant comprises between 1 and 4% 1,8-cineole. In one aspect, the essential oil derived from a hybrid mint plant comprises between 1 and 3% 1,8-cineole. In one aspect, the essential oil derived from a hybrid mint plant comprises between 1 and 2% 1,8-cineole.

In one aspect, the essential oil derived from a hybrid mint plant comprises between 1 and 3% 3-octanol, determined by calculation of the relative peak areas of a gas chromatograph of the at least one essential oil. In one aspect, the essential oil derived from a hybrid mint plant comprises between 1 and 2.5% 3-octanol. In one aspect, the essential oil derived from a hybrid mint plant comprises between 1 and 2% 3-octanol. In one aspect, the essential oil derived from a hybrid mint plant comprises between 1 and 1.5% 3-octanol.

In one aspect, the essential oil derived from a hybrid mint plant comprises between 1 and 1.5% menthone, determined by calculation of the relative peak areas of a gas chromatograph of the at least one essential oil.

In one aspect, the essential oil derived from a hybrid mint plant comprises between 0.5 and 5% iso-menthone, determined by calculation of the relative peak areas of a gas chromatograph of the at least one essential oil. In one aspect, the essential oil derived from a hybrid mint plant comprises between 0.5 and 4.5% iso-menthone. In one aspect, the essential oil derived from a hybrid mint plant comprises between 0.5 and 4.5% iso-menthone. In one aspect, the essential oil derived from a hybrid mint plant comprises between 0.5 and 4% iso-menthone. In one aspect, the essential oil derived from a hybrid mint plant comprises between 0.5 and 3.5% iso-menthone. In one aspect, the essential oil derived from a hybrid mint plant comprises between 0.5 and 3% iso-menthone. In one aspect, the essential oil derived from a hybrid mint plant comprises between 0.5 and 2.5% iso-menthone. In one aspect, the essential oil derived from a hybrid mint plant comprises between 0.5 and 2% iso-menthone. In one aspect, the essential oil derived from a hybrid mint plant comprises between 0.5 and 1.5% iso-menthone. In one aspect, the essential oil derived from a hybrid mint plant comprises between 0.5 and 1% iso-menthone.

In one aspect, the essential oil derived from a hybrid mint plant comprises between 0.5 and 20% l-menthyl acetate, determined by calculation of the relative peak areas of a gas chromatograph of the at least one essential oil. In one aspect, the essential oil derived from a hybrid mint plant comprises between 0.5 and 15% l-menthyl acetate. In one aspect, the essential oil derived from a hybrid mint plant comprises between 0.5 and 10% l-menthyl acetate. In one aspect, the essential oil derived from a hybrid mint plant comprises between 0.5 and 9% l-menthyl acetate. In one aspect, the essential oil derived from a hybrid mint plant comprises between 0.5 and 8% l-menthyl acetate. In one aspect, the essential oil derived from a hybrid mint plant comprises between 0.5 and 7% l-menthyl acetate. In one aspect, the essential oil derived from a hybrid mint plant comprises between 0.5 and 6% l-menthyl acetate. In one aspect, the essential oil derived from a hybrid mint plant comprises between 0.5 and 5% l-menthyl acetate. In one aspect, the essential oil derived from a hybrid mint plant comprises between 0.5 and 4% l-menthyl acetate. In one aspect, the essential oil derived from a hybrid mint plant comprises between 0.5 and 3% l-menthyl acetate. In one aspect, the essential oil derived from a hybrid mint plant comprises between 0.5 and 2% l-menthyl acetate. In one aspect, the essential oil derived from a hybrid mint plant comprises between 0.5 and 1% l-menthyl acetate.

In one aspect, the essential oil derived from a hybrid mint plant comprises between 1 and 4% neo-menthol, determined by calculation of the relative peak areas of a gas chromatograph of the at least one essential oil. In one aspect, the essential oil derived from a hybrid mint plant comprises between 1 and 3.5% neo-menthol. In one aspect, the essential oil derived from a hybrid mint plant comprises between 1 and 3% neo-menthol. In one aspect, the essential oil derived from a hybrid mint plant comprises between 1 and 2.5% neo-menthol. In one aspect, the essential oil derived from a hybrid mint plant comprises between 1 and 2% neo-menthol. In one aspect, the essential oil derived from a hybrid mint plant comprises between 1 and 1.5% neo-menthol.

In one aspect, the essential oil derived from a hybrid mint plant comprises between 0.5 and 2% β-caryophyllene, determined by calculation of the relative peak areas of a gas chromatograph of the at least one essential oil. In one aspect, the essential oil derived from a hybrid mint plant comprises between 0.5 and 1.5% 3-caryophyllene. In one aspect, the essential oil derived from a hybrid mint plant comprises between 0.5 and 1% β-caryophyllene.

In one aspect, the essential oil derived from a hybrid mint plant comprises between 0.5 and 2% pulegone, determined by calculation of the relative peak areas of a gas chromatograph of the at least one essential oil. In one aspect, the essential oil derived from a hybrid mint plant comprises between 0.5 and 1.5% pulegone. In one aspect, the essential oil derived from a hybrid mint plant comprises between 0.5 and 1% pulegone.

In one aspect, the essential oil derived from a hybrid mint plant comprises between 0.5 and 2% germacrene-D, determined by calculation of the relative peak areas of a gas chromatograph of the at least one essential oil. In one aspect, the essential oil derived from a hybrid mint plant comprises between 0.5 and 1.5% germacrene-D. In one aspect, the essential oil derived from a hybrid mint plant comprises between 0.5 and 1% germacrene-D.

In one aspect, the essential oil derived from a hybrid mint plant comprises between 1 and 4% dihydrocarvone, determined by calculation of the relative peak areas of a gas chromatograph of the at least one essential oil. In one aspect, the essential oil derived from a hybrid mint plant comprises between 1 and 3.5% dihydrocarvone. In one aspect, the essential oil derived from a hybrid mint plant comprises between 1 and 3% dihydrocarvone. In one aspect, the essential oil derived from a hybrid mint plant comprises between 1 and 2.5% dihydrocarvone. In one aspect, the essential oil derived from a hybrid mint plant comprises between 1 and 1% dihydrocarvone. In one aspect, the essential oil derived from a hybrid mint plant comprises between 1 and 1.5% dihydrocarvone.

In one aspect, the ratio of l-menthone to l-menthol in the essential oil derived from a hybrid mint plant is from 8:1 to 1:7.

In one aspect, the at least one essential oil is an essential oil described in Tables 1 through 13.

In one aspect, the essential oil derived from a hybrid mint plant comprises 1 to 4% l-limonene, 0.5 to 7% 1,8-cineole, 2 to 62% l-menthone, 0 to 50% menthofuran, 0.5 to 5% isomenthone, 0.5 to 20% l-menthyl acetate, 1 to 4% neo-menthol, 7 to 65% l-menthol, and 0 to 2% pulegone, wherein the percentage is determined by calculation of the relative peak areas of a gas chromatograph of the at least one essential oil. In some aspects, the essential oil derived from a hybrid mint plant further comprises 0.5 to 2% β-caryophyllene. In some aspects, the essential oil derived from a hybrid mint plant further comprises 0.5 to 2% germacrene-D.

In one aspect, the essential oil derived from a hybrid mint plant comprises 0.5 to 2% a-pinene, 1 to 2% β-pinene, 1 to 4% l-limonene, 0.5 to 7% 1,8-cineole, 2 to 62% l-menthone, 0 to 50% menthofuran, 0.5 to 5% isomenthone, 0.5 to 20% l-menthyl acetate, 1 to 4% neo-menthol, 7 to 65% l-menthol, and 0 to 2% pulegone, wherein the percentage is determined by calculation of the relative peak areas of a gas chromatograph of the at least one essential oil. In some aspects, the essential oil derived from a hybrid mint plant further comprises 0.5 to 2% β-caryophyllene.

In one aspect, the essential oil derived from a hybrid mint plant comprises 20 to 40% d-limonene, 0.5 to 7% 1,8-cineole, 1 to 3% 3-octanol, 1 to 3% menthone, 1 to 4% dihydrocarvone, and 20 to 40% l-carvone.

In one aspect, the at least one essential oil comprises the essential oil extracted from the hybrid mint plant designated 13-S12-2. In an alternate aspect, the at least one essential oil comprises the essential oil extracted from the hybrid mint plant designated 13-39-9. In an alternate aspect, the at least one essential oil comprises the essential oil extracted from the hybrid mint plant designated 13-A36-13. In an alternate aspect, the at least one essential oil comprises the essential oil extracted from the hybrid mint plant designated 07-A3-11. In an alternate aspect, the at least one essential oil comprises the essential oil extracted from the hybrid mint plant designated 09-6-2. In an alternate aspect, the at least one essential oil comprises the essential oil extracted from the hybrid mint plant designated 11-A35-3. In an alternate aspect, the at least one essential oil comprises the essential oil extracted from the hybrid mint plant designated 08-6-10. In an alternate aspect, the at least one essential oil comprises the essential oil extracted from the hybrid mint plant designated 08-A20-3. In an alternate aspect, the at least one essential oil comprises the essential oil extracted from the hybrid mint plant designated 05-19-1. In an alternate aspect, the at least one essential oil comprises the essential oil extracted from the hybrid mint plant designated 07-A3-5A. In an alternate aspect, the at least one essential oil comprises the essential oil extracted from the hybrid mint plant designated 14-41-16. In an alternate aspect, the at least one essential oil comprises the essential oil extracted from the hybrid mint plant designated 14-27-71. In an alternate aspect, the at least one essential oil comprises the essential oil extracted from the hybrid mint plant designated 14-27-89.

In one aspect, the essential oil is derived from the hybrid mint plant disclosed in U.S. Patent No. PP25,108 P3. In an alternate aspect, the essential oil is derived from the hybrid mint plant disclosed in U.S. Patent No. PP25,109 P3. In an alternate aspect, the essential oil is derived from the hybrid mint plant disclosed in U.S. Patent No. PP25,138 P3. In an alternate aspect, the essential oil is derived from the hybrid mint plant disclosed in U.S. Patent No. PP25,415 P3. In an alternate aspect, the essential oil is derived from the hybrid mint plant disclosed in U.S. Patent No. PP25,439 P3. In an alternate aspect, the essential oil is derived from the hybrid mint plant disclosed in U.S. Patent No. PP25,440 P3. In an alternate aspect, the essential oil is derived from the hybrid mint plant disclosed in U.S. Patent No. PP25,441 P3. In an alternate aspect, the essential oil is derived from the hybrid mint plant disclosed in U.S. Patent No. PP27,602 P3. In an alternate aspect, the essential oil is derived from the hybrid mint plant disclosed in U.S. Patent No. PP27,603 P3. In an alternate aspect, the essential oil is derived from the hybrid mint plant disclosed in U.S. Patent No. PP27,604 P3.

In one aspect, the at least one essential oil comprises the essential oil disclosed in U.S. Patent No. PP25,108 P3. In an alternate aspect, the at least one essential oil comprises the essential oil disclosed in U.S. Patent No. PP25,109 P3. In an alternate aspect, the at least one essential oil comprises the essential oil disclosed in U.S. Patent No. PP25,138 P3. In an alternate aspect, the at least one essential oil comprises the essential oil disclosed in U.S. Patent No. PP25,415 P3. In an alternate aspect, the at least one essential oil comprises the essential oil disclosed in U.S. Patent No. PP25,439 P3. In an alternate aspect, the at least one essential oil comprises the essential oil disclosed in U.S. Patent No. PP25,440 P3. In an alternate aspect, the at least one essential oil comprises the essential oil disclosed in U.S. Patent No. PP25,441 P3. In an alternate aspect, the at least one essential oil comprises the essential oil disclosed in U.S. Patent No. PP27,602 P3. In an alternate aspect, the at least one essential oil comprises the essential oil disclosed in U.S. Patent No. PP27,603 P3. In an alternate aspect, the at least one essential oil comprises the essential oil disclosed in U.S. Patent No. PP27,604 P3.

In one aspect, the plant extract further comprises at least one extract derived from a commercial mint plant, wherein the extract comprises an essential oil. Non-limiting examples of commercial mint plant extracts include: an extract derived from Piperita Idaho, Pepper Mint oil, an extract derived from Piperita India, an extract derived from Piperita Mid West, an extract derived from Piperita Cascade, an extract derived from Piperita Terpeneless, an extract derived from Piperita Yakima Single Cut, an extract derived from Spicata.

In one aspect, the commercial mint plant is Native Spearmint. In an alternate aspect, the commercial mint plant is Scotch Spearmint. In an alternate aspect, the commercial mint plant is Black Mitcham. In an alternate embodiment, the commercial mint plant is M. arvensis. In an alternate embodiment, the commercial mint plant is M. piperna.

In one aspect, the commercial mint plant is selected from the mint plants disclosed in U.S. Patent No. PP25,108 P3. In an alternate aspect, the commercial mint plant is selected from the mint plants disclosed in U.S. Patent No. PP25,109 P3. In an alternate aspect, the commercial mint plant is selected from the mint plants disclosed in U.S. Pat. No. PP25,138 P3. In an alternate aspect, the commercial mint plant is selected from the mint plants disclosed in U.S. Patent No. PP25,415 P3. In an alternate aspect, the commercial mint plant is selected from the mint plants disclosed in U.S. Pat. No. PP25,439 P3. In an alternate aspect, the commercial mint plant is selected from the mint plants disclosed in U.S. Patent No. PP25,440 P3. In an alternate aspect, the commercial mint plant is selected from the mint plants disclosed in U.S. Patent No. PP25,441 P3. In an alternate aspect, the commercial mint plant is selected from the mint plants disclosed in U.S. Patent No. PP27,602 P3. In an alternate aspect, the commercial mint plant is selected from the mint plants disclosed in U.S. Patent No. PP27,603 P3. In an alternate aspect, the commercial mint plant is selected from the mint plants disclosed in U.S. Pat. No. PP27,604 P3.

In one aspect, the concentration of d-limonene in the essential oil derived from a commercial mint plant is from 5 to 20%, determined by calculation of the relative peak areas of a gas chromatograph of the at least one essential oil. In one aspect, the concentration of d-limonene in the essential oil derived from a commercial mint plant is from 5 to 15%, alternatively, from 5 to 10%, determined by calculation of the relative peak areas of a gas chromatograph of the at least one essential oil.

In one aspect, the essential oil derived from a commercial mint plant comprises between 1 and 4% l-limonene, determined by calculation of the relative peak areas of a gas chromatograph of the at least one essential oil. In one aspect, the essential oil derived from a commercial mint plant comprises between 1 and 3.5% l-limonene. In one aspect, the essential oil derived from a commercial mint plant comprises between 1 and 3% 1-limonene. In one aspect, the essential oil derived from a commercial mint plant comprises between 1 and 2.5% l-limonene. In one aspect, the essential oil derived from a commercial mint plant comprises between 1 and 1% l-limonene. In one aspect, the essential oil derived from a commercial mint plant comprises between 1 and 1.5% l-limonene.

In one aspect, the essential oil derived from a commercial mint plant comprises between 1 and 7% 1,8-cineole, determined by calculation of the relative peak areas of a gas chromatograph of the at least one essential oil. In one aspect, the essential oil derived from a commercial mint plant comprises between 1 and 6% 1,8-cineole. In one aspect, the essential oil derived from a commercial mint plant comprises between 1 and 5% 1,8-cineole. In one aspect, the essential oil derived from a commercial mint plant comprises between 1 and 4% 1,8-cineole. In one aspect, the essential oil derived from a commercial mint plant comprises between 1 and 3% 1,8-cineole. In one aspect, the essential oil derived from a commercial mint plant comprises between 1 and 2% 1,8-cineole.

In one aspect, the essential oil derived from a commercial mint plant comprises between 1 and 3% 3-octanol, determined by calculation of the relative peak areas of a gas chromatograph of the at least one essential oil. In one aspect, the essential oil derived from a commercial mint plant comprises between 1 and 2.5% 3-octanol. In one aspect, the essential oil derived from a commercial mint plant comprises between 1 and 2% 3-octanol. In one aspect, the essential oil derived from a commercial mint plant comprises between 1 and 1.5% 3-octanol.

In one aspect, the essential oil derived from a commercial mint plant comprises between 1 and 1.5% menthone, determined by calculation of the relative peak areas of a gas chromatograph of the at least one essential oil.

In one aspect, the concentration of l-menthone in the essential oil derived from a commercial mint plant is from 7 to 30%, determined by calculation of the relative peak areas of a gas chromatograph of the at least one essential oil. In one aspect, the concentration of carvone in the essential oil derived from a commercial mint plant is from 7 to 20%, alternatively, from 7 to 10%, alternatively, from 7 to 15%, alternatively, from 7 to 10%.

In one aspect, the concentration of menthofuran in the essential oil derived from a commercial mint plant is from 0 to 5%, determined by calculation of the relative peak areas of a gas chromatograph of the at least one essential oil. In one aspect, the concentration of menthofuran in the essential oil derived from a commercial mint plant is from 0 to 3%, alternatively, from 0 to 2%, alternatively, from 0 to 1%, alternatively, from 0 to 0.5%.

In one aspect, the essential oil derived from a commercial mint plant comprises between 2 and 4% iso-menthone, determined by calculation of the relative peak areas of a gas chromatograph of the at least one essential oil. In one aspect, the essential oil derived from a commercial mint plant comprises between 2 and 3.5% iso-menthone. In one aspect, the essential oil derived from a commercial mint plant comprises between 2 and 3% iso-menthone. In one aspect, the essential oil derived from a commercial mint plant comprises between 2 and 2.5% iso-menthone.

In one aspect, the essential oil derived from a commercial mint plant comprises between 2 and 6% l-menthyl acetate, determined by calculation of the relative peak areas of a gas chromatograph of the at least one essential oil. In one aspect, the essential oil derived from a commercial mint plant comprises between 2 and 5.5% l-menthyl acetate. In one aspect, the essential oil derived from a commercial mint plant comprises between 2 and 5% l-menthyl acetate. In one aspect, the essential oil derived from a commercial mint plant comprises between 2 and 4.5% l-menthyl acetate. In one aspect, the essential oil derived from a commercial mint plant comprises between 2 and 4% l-menthyl acetate. In one aspect, the essential oil derived from a commercial mint plant comprises between 2 and 3.5% l-menthyl acetate. In one aspect, the essential oil derived from a commercial mint plant comprises between 2 and 3% l-menthyl acetate. In one aspect, the essential oil derived from a commercial mint plant comprises between 2 and 2.5% l-menthyl acetate.

In one aspect, the essential oil derived from a commercial mint plant comprises between 3 and 6% neo-menthol, determined by calculation of the relative peak areas of a gas chromatograph of the at least one essential oil. In one aspect, the essential oil derived from a commercial mint plant comprises between 3 and 5.5% neo-menthol. In one aspect, the essential oil derived from a commercial mint plant comprises between 3 and 5% neo-menthol. In one aspect, the essential oil derived from a commercial mint plant comprises between 3 and 4.5% neo-menthol. In one aspect, the essential oil derived from a commercial mint plant comprises between 3 and 4% neo-menthol. In one aspect, the essential oil derived from a commercial mint plant comprises between 3 and 3.5% neo-menthol.

In one aspect, the essential oil derived from a commercial mint plant comprises between 0.5 and 1% β-caryophyllene, determined by calculation of the relative peak areas of a gas chromatograph of the at least one essential oil. In one aspect, the essential oil derived from a commercial mint plant comprises between 0.5 and 0.9% β-caryophyllene. In one aspect, the essential oil derived from a commercial mint plant comprises between 0.5 and 0.8% ρ-caryophyllene.

In one aspect, the concentration of l-menthol in the essential oil derived from a commercial mint plant is from 40 to 80%, determined by calculation of the relative peak areas of a gas chromatograph of the at least one essential oil. In one aspect, the concentration of l-menthol in the essential oil derived from a commercial mint plant is from 40 to 70%, alternatively, from 40 to 60%, alternatively, from 40 to 50%.

In one aspect, the essential oil derived from a commercial mint plant comprises between 0.5 and 2% pulegone, determined by calculation of the relative peak areas of a gas chromatograph of the at least one essential oil. In one aspect, the essential oil derived from a commercial mint plant comprises between 0.5 and 1.5% pulegone. In one aspect, the essential oil derived from a commercial mint plant comprises between 0.5 and 1% pulegone.

In one aspect, the essential oil derived from a commercial mint plant comprises between 0.5 and 2% germacrene-D, determined by calculation of the relative peak areas of a gas chromatograph of the at least one essential oil. In one aspect, the essential oil derived from a commercial mint plant comprises between 0.5 and 1.5% germacrene-D. In one aspect, the essential oil derived from a commercial mint plant comprises between 0.5 and 1% germacrene-D.

In one aspect, the essential oil derived from a commercial mint plant comprises between 0.5 and 2% dihydrocarvone, determined by calculation of the relative peak areas of a gas chromatograph of the at least one essential oil. In one aspect, the essential oil derived from a commercial mint plant comprises between 0.5 and 1.5% dihydrocarvone. In one aspect, the essential oil derived from a commercial mint plant comprises between 0.5 and 1% dihydrocarvone.

In one aspect, the concentration of l-carvone in the essential oil derived from a commercial mint plant is from 60 to 80%, determined by calculation of the relative peak areas of a gas chromatograph of the at least one essential oil. In one aspect, the concentration of l-carvone in the essential oil derived from a commercial mint plant is from 60 to 70%, alternatively, from 60 to 65%.

In one aspect, the ratio of l-menthone to l-menthol in the essential oil derived from a commercial mint plant is from 1:2 to 1:10.

In one aspect, the at least one essential oil is an essential oil described in Tables 14 through 18.

In one aspect, the essential oil derived from a commercial mint plant comprises 5 to 20% d-limonene, 1 to 7% 1,8-cineole, 1 to 3% 3-octanol, 1 to 1.5% menthone, 0.5 to 2% dihydrocarvone, and 60 to 80% l-carvone.

In one aspect, the essential oil derived from a commercial mint plant comprises 1 to 4% l-limonene, 1 to 7% 1,8-cineole, 1 to 1.5% menthone, 0 to 5% menthofuran, 2 to 4% iso-menthone, 2 to 6% l-menthyl acetate, 3 to 6% neo-menthol, 0.5 to 1% β-caryophyllene, 40 to 80% l-menthol, 0.5 to 2% pulegone, and 0.5 to 2% germacrene-D.

One of ordinary skill in the art can readily appreciate that any essential oil derived from a hybrid mint plant described herein may be replicated by combining the individual volatile aroma compounds of the essential oil. For example, by way of illustration, a composition replicating an essential oil derived from a hybrid mint plant described herein may be replicated by combining individual volatile aroma compounds as described in any one of the Tables 1 through 13.

Accordingly, in one aspect, the composition comprises 1 to 4% l-limonene, 0.5 to 7% 1,8-cineole, 2 to 62% l-menthone, 0 to 50% menthofuran, 0.5 to 5% isomenthone, 0.5 to 20% l-menthyl acetate, 1 to 4% neo-menthol, 7 to 65% l-menthol, and 0 to 2% pulegone, wherein the percentage is determined by calculation of the relative peak areas of a gas chromatograph of the at least one essential oil. In some aspects, the essential oil derived from a hybrid mint plant further comprises 0.5 to 2% β-caryophyllene. In some aspects, the composition further comprises 0.5 to 2% germacrene-D.

In one aspect, the composition comprises 0.5 to 2% a-pinene, 1 to 2% β-pinene, 1 to 4% l-limonene, 0.5 to 7% 1,8-cineole, 2 to 62% l-menthone, 0 to 50% menthofuran, 0.5 to 5% isomenthone, 0.5 to 20% l-menthyl acetate, 1 to 4% neo-menthol, 7 to 65% 1-menthol, and 0 to 2% pulegone, wherein the percentage is determined by calculation of the relative peak areas of a gas chromatograph of the at least one essential oil. In some aspects, the composition further comprises 0.5 to 2% β-caryophyllene.

In one aspect, the composition comprises 20 to 40% d-limonene, 0.5 to 7% 1,8-cineole, 1 to 3% 3-octanol, 1 to 3% menthone, 1 to 4% dihydrocarvone, and 20 to 40% l-carvone.

Similarly, one of ordinary skill in the art can readily appreciate that any essential oil derived from a commercial mint plant described herein may be replicated by combining the individual volatile aroma compounds of the essential oil. For example, by way of illustration, a composition replicating an essential oil derived from a commercial mint plant described herein may be replicated by combining individual volatile aroma compounds as described in any one of the Tables 14 through 18.

Accordingly, in one aspect, the composition comprises 5 to 20% d-limonene, 1 to 7% 1,8-cineole, 1 to 3% 3-octanol, 1 to 1.5% menthone, 0.5 to 2% dihydrocarvone, and 60 to 80% l-carvone.

In one aspect, the composition comprises 1 to 4% l-limonene, 1 to 7% 1,8-cineole, 1 to 1.5% menthone, 0 to 5% menthofuran, 2 to 4% iso-menthone, 2 to 6% l-menthyl acetate, 3 to 6% neo-menthol, 0.5 to 1% β-caryophyllene, 40 to 80% l-menthol, 0.5 to 2% pulegone, and 0.5 to 2% germacrene-D.

### The at Least One Cooling Compound.

In one aspect, the at least one cooling compound comprises amides of formula (I):

A¹-[B]—CO—NR³—(CH2)pA² (I)

in which
A¹ stands for any substituted aryl, heteroaryl, or cycloaryl residue,
B for an OCRbRb, CHRc-CHRd, CRe=CRf, or a cycloaryl residue,
p for numbers from 1-3,
R^{a} for any substituted alkyl, heteroalkyl, alkenyl, heteroalkenyl, aryl, aralkyl, heteroaryl cyclo-, or heterocycloalkyl residue with 1-20 carbon atoms,
R^{b}, R^{e}, R^{d}, R^{e}, and R^{f} independently stand for hydrogen or an alkyl residue with 1-4 carbon atoms, and
A² stands for any substituted five- or six-membered heteroaryl residue having at least one heteroatom from the group of nitrogen, oxygen, and sulphur,

as well as their salts,

In one aspect, the at least one cooling compound comprises acyclic carboxamides.

In one aspect, the at least one cooling compound comprises N-substituted p-menthane carboxamides.

In one aspect, the at least one cooling compound is selected from the group consisting of WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (1R,2S,5R)—N-(4-Methoxyphenyl)-p-menthanecarboxamide, FEMA 4681; WS-27 (N-Ethyl-2,2-diisopropylbutanamide), FEMA 4557; N-Cyclopropyl-5-methyl-2-isopropylcyclohexanecarboxamide, FEMA 4693, WS-116 (N-(1,1-Dimethyl-2-hydroxyethyl)-2,2-diethylbutanamide), N-(1,1-Dimethyl-2-hydroxyethyl)2,2-diethylbutanamide, FEMA 4603, Menthoxyethanol, FEMA 4154, N-(4-cyanomethylphenyl)-p-menthanecarboxamide, FEMA 4496; N-(2-(Pyridin-2-yl)ethyl)-3-p-menthanecarboxamide, FEMA 4549; N-(2-Hydroxyethyl)-2-isopropyl-2,3-dimethylbutanamide, FEMA 4602 and (also N-(4-(carbamoylmethyl)phenyl)-menthylcarboxamide, FEMA 4684; (1R,2S,5R)—N-(4-Methoxyphenyl)-p-menthanecarboxamide (WS-12), FEMA 4681; (2S,5R)—N-[4-(2-Amino-2-oxoethyl)phenyl]-p-menthanecarboxamide, FEMA 4684; and N-Cyclopropyl-5-methyl-2-isopropylcyclohexanecarbonecarboxamide, FEMA 4693; 2-[(2-p-Menthoxy)ethoxy] ethanol, FEMA 4718; (2,6-Diethyl-5-isopropyl-2-methyltetrahydropyran, FEMA 4680); trans-4-tert-Butylcyclohexanol, FEMA 4724; 2-(p-tolyloxy)-N-(1H-pyrazol-5-yl)-N-((thiophen-2-yl)methyl)acetamide, FEMA 4809; Menthone glycerol ketal, FEMA 3807; Menthone glycerol ketal; (−)-Menthoxypropane-1,2-diol; 3-(1-Menthoxy)-2-methylpropane-1,2-diol, FEMA 3849; Isopulegol; (+)-cis & (−)-trans p-Menthane-3,8-diol, Ratio ˜62:38, FEMA 4053; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3.6,9-trioxadecanoate; (1R,2S,5R)-3-menthyl (2-hydroxyethoxy)acetate; (1R,2S,5R)-menthyl 11-hydroxy-3,6,9-trioxaundecanoate; Cubebol, FEMA 4497; N-(4-cyanomethylphenyl) p-menthanecarboxamide, FEMA 4496; 2-isopropyl-5-methylcyclohexyl 4-(dimethylamino)-4-oxobutanoate, FEMA 4230; N-(4-cyanomethylphenyl) p-menthanecarboxamide, FEMA 4496; N-(2-pyridin-2-ylethyl) p-; menthanecarboxamide, FEMA 4549, Menthyl lactate, FEMA 3748; 6-isopropyl-3,9-dimethyl-1,4-dioxaspiro[4.5]decan-2-one, FEMA 4285; N-benzo[1,3] dioxol-5-yl-3-p-menthanecarboxamide; N-(1-isopropyl-1,2-dimethylpropyl)-1,3-benzodioxole-5-carboxamide; N—(R)-2-oxotetrahydrofuran-3-yl-(1R,2S,5R)-p-menthane-3-carboxamide; mixture of 2,2,5,6,6-pentamethyl-2,3,6,6a-tetrahydropentalen-3 a(1H)-ol and 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide, FEMA 4549; (2S,5R)-2-isopropyl-5-methyl-N-(2-(pyridin-4-yl)ethyl)cyclohexanecarboxamide; N-(4-cyanomethylphenyl) p-menthanecarboxamide, FEMA 4496; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]benzenesulfonamide; 4-cyano-N-phenyl-N-[2-(pyridin-2-yl)ethyl]benzenesulfonamid; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol, 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(14 sopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-methan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl and isobornyl; menthyl oxamate derivatives; menthyl 3-oxocarboxylic acid esters; N alpha-(Menthanecarbonyl)amino acid amides; p-menthane carboxamide and WS-23 analogs; (−)-(1R,2R,4S)-dihydroumbellulol; p-menthane alkyloxy amides; cyclohexane derivatives; butone derivatives; a mixture of 3-menthoxy-1-propanol and 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; and combinations thereof.

In one aspect, the at least one cooling compound is 2-(4-methylphenoxy)-N-(1H-pyrazol-3-yl)-N-(2-thienylmethyl) acetamide.

Examples of cooling compounds suitable to formulate oral and/or dental care products according to certain aspects presented herein are disclosed in U.S. Pat. No. 9,732,071.

Examples of cooling compounds suitable to formulate oral and/or dental care products according to certain aspects presented herein are disclosed in U.S. Patent Application Publication No. 20170087199.

Examples of cooling compounds suitable to formulate oral and/or dental care products according to certain aspects presented herein are disclosed in U.S. Patent Application Publication No. 20160339026.

Examples of cooling compounds suitable to formulate oral and/or dental care products according to certain aspects presented herein are disclosed in U.S. Patent Application Publication No. 20150376136.

Examples of cooling compounds suitable to formulate oral and/or dental care products according to certain aspects presented herein are disclosed in U.S. Pat. No. 9,394,287.

Examples of cooling compounds suitable to formulate oral and/or dental care products according to certain aspects presented herein are disclosed in International Patent Application Publication No. WO2014090293.

Examples of cooling compounds suitable to formulate oral and/or dental care products according to certain aspects presented herein are disclosed in International Patent Application Publication No. WO2012061698.

Examples of cooling compounds suitable to formulate oral and/or dental care products according to certain aspects presented herein are disclosed in U.S. Patent Application Publication No. 2010007608.

Examples of cooling compounds suitable to formulate oral and/or dental care products according to certain aspects presented herein are disclosed in U.S. Patent Application Publication No. 20080319055.

Examples of cooling compounds suitable to formulate oral and/or dental care products according to certain aspects presented herein are disclosed in U.S. Patent Application Publication No. 20080311232.

Examples of cooling compounds suitable to formulate oral and/or dental care products according to certain aspects presented herein are disclosed in U.S. Patent Application Publication No. 20090054520.

Examples of cooling compounds suitable to formulate oral and/or dental care products according to certain aspects presented herein are disclosed in U.S. Patent Application Publication No. 20080177800.

Examples of cooling compounds suitable to formulate oral and/or dental care products according to certain aspects presented herein are disclosed in U.S. Patent Application Publication No. 20080096969.

Examples of cooling compounds suitable to formulate oral and/or dental care products according to certain aspects presented herein are disclosed in International Patent Application Publication No. WO2010128026.

Examples of cooling compounds suitable to formulate oral and/or dental care products according to certain aspects presented herein are disclosed in International Patent Application Publication No. WO2011061330.

Examples of cooling compounds suitable to formulate oral and/or dental care products according to certain aspects presented herein are disclosed in International Patent Application Publication No. WO2011138696.

Examples of cooling compounds suitable to formulate oral and/or dental care products according to certain aspects presented herein are disclosed in U.S. Pat. No. 8,377,422.

Examples of cooling compounds suitable to formulate oral and/or dental care products according to certain aspects presented herein are disclosed in International Patent Application Publication No. WO2013033501.

Examples of cooling compounds suitable to formulate oral and/or dental care products according to certain aspects presented herein are disclosed in U.S. Patent Application Publication No. 20130216486.

Examples of cooling compounds suitable to formulate oral and/or dental care products according to certain aspects presented herein are disclosed in U.S. Pat. No. 7,935,848.

Examples of cooling compounds suitable to formulate oral and/or dental care products according to certain aspects presented herein are disclosed in U.S. Patent Application Publication No. 20130345300.

Examples of cooling compounds suitable to formulate oral and/or dental care products according to certain aspects presented herein are disclosed in International Patent Application Publication No. WO2013080830.

Examples of cooling compounds suitable to formulate oral and/or dental care products according to certain aspects presented herein are disclosed in International Patent Application Publication No. WO2014010657.

Examples of cooling compounds suitable to formulate oral and/or dental care products according to certain aspects presented herein are disclosed in U.S. Patent Application Publication No. 20140219930.

Examples of cooling compounds suitable to formulate oral and/or dental care products according to certain aspects presented herein are disclosed in U.S. Patent Application Publication No. 20150139918.

### The at Least One Flavor Compound.

In one aspect, at least one flavor compound is an extract selected from the group consisting of: linseed, Eucalyptus, Rosemary, Marjoram, German Chamomille (Blue), Roman Chamomile, Laurel, Basil, Angelica root, Angelica seed, Cilantro, Coriander, Dill, Thyme, Parsley, Sage officinalis, Clary sage, Spanish sage, Myrrha, Clove, Ginger, Cardamom, Mate, Star anise, Valerian, Pepper, Pine, Oregano, Nutmeg, Lovage, Juniper, Gentiane, Fenugreek, and Lemongrass. The extract may be any form, including, but not limited to an oil, an oleoresin, and the like.

TABLE 1
An Essential Oil Derived From a Hybrid
Mint Plant According to One
Aspect Presented Herein.

| Component | % |
|---|---|
| d-linonene | 30.3 |
| 1,8-cineole | <1.0 |
| 3-octanol | 2.4 |
| menthone | 1.3 |
| dihydrocarvone | 3.0 |
| l-carvone | 31.5 |

TABLE 2
An Essential Oil Derived From a Hybrid
Mint Plant According to One
Aspect Presented Herein.

| Component | % |
|---|---|
| l-linonene | 3.2 |
| 1,8-cineole | 5.6 |
| l-menthone | 15.5 |
| menthofuran | 28.6 |
| isomenthone | 1.5 |
| l-menthyl acetate | 4.9 |
| neo-menthol | 2.1 |
| β-caryophyllene | 0.6 |
| 1-menthol | 26.2 |
| pulegone | 0.8 |
| germacrene-D | 1.2 |

TABLE 3
An Essential Oil Derived From a Hybrid
Mint Plant According to One
Aspect Presented Herein.

| Component | % |
|---|---|
| l-linonene | 1.7 |
| 1,8-cineole | 4.9 |
| l-menthone | 19.4 |
| menthofuran | 4.2 |
| isomenthone | 3.1 |
| l-menthyl acetate | 5.3 |
| 1-menthol | 44.6 |
| pulegone | 2.128 |

TABLE 4
An Essential Oil Derived From a Hybrid
Mint Plant According to One
Aspect Presented Herein.

| Component | % |
|---|---|
| l-linonene | 2.2 |
| 1,8-cineole | <1.0 |
| l-menthone | 28.4 |
| menthofuran | <1.0 |
| isomenthone | 3.1 |
| l-menthyl acetate | <1.0 |
| 1-menthol | 59.1 |
| pulegone | 0 |

TABLE 5
An Essential Oil Derived From a Hybrid
Mint Plant According to One
Aspect Presented Herein.

| Component | % |
|---|---|
| l-linonene | 3.7 |
| 1,8-cineole | <1.0 |
| l-menthone | 31.9 |
| menthofuran | 2.3 |
| isomenthone | 1.2 |
| l-menthyl acetate | 5.5 |
| 1-menthol | 39.1 |
| pulegone | <1.0 |

TABLE 6
An Essential Oil Derived From a Hybrid
Mint Plant According to One
Aspect Presented Herein.

| Component | % |
|---|---|
| l-linonene | 1.5 |
| 1,8-cineole | <1.0 |
| l-menthone | 19.9 |
| menthofuran | <1.0 |
| isomenthone | 4.5 |
| l-menthyl acetate | 2.1 |
| 1-menthol | 62.6 |
| pulegone | <1.0 |

TABLE 7
An Essential Oil Derived From a Hybrid Mint
Plant According to One Aspect Presented Herein.

| Component | % |
|---|---|
| α-pinene | <1.0 |
| β-pinene | <1.0 |
| l-linonene | 1.4 |
| 1,8-cineole | 4.2 |
| l-menthone | 10.0 |
| menthofuran | 20.7 |
| isomenthone | 1.3 |
| l-menthyl acetate | 7.8 |
| neo-menthol | 4.0 |
| β-caryophyllene | 1.6 |
| 1-menthol | 44.2 |
| pulegone | <1.0 |
| germacrene-D | <1.0 |

TABLE 8
An Essential Oil Derived From a Hybrid Mint
Plant According to One Aspect Presented Herein.

| Component | % |
|---|---|
| l-linonene | 1.3 |
| 1,8-cineole | <1.0 |
| l-menthone | 42.2 |
| menthofuran | 3.8 |
| isomenthone | 2.5 |
| l-menthyl acetate | 4.3 |
| 1-menthol | 35.4 |
| pulegone | <1.0 |

TABLE 9
An Essential Oil Derived From a Hybrid Mint
Plant According to One Aspect Presented Herein.

| Component | % |
|---|---|
| α-pinene | 0.84 |
| β-pinene | 1.17 |
| l-linonene | 2.24 |
| 1,8-cineole | 3.71 |
| l-menthone | 2.47 |
| menthofuran | 44.02 |
| isomenthone | 0.58 |
| l-menthyl acetate | 19.67 |
| neo-menthol | 1.9 |
| β-caryophyllene | 1.6 |
| 1-menthol | 18.3 |
| pulegone | <1.0 |
| germacrene-D | <1.0 |

TABLE 10
An Essential Oil Derived From a Hybrid Mint
Plant According to One Aspect Presented Herein.

| Component | % |
|---|---|
| l-linonene | 1.6 |
| 1,8-cineole | <1.0 |
| l-menthone | 44.2 |
| menthofuran | <1.0 |
| isomenthone | 3.1 |
| l-menthyl acetate | 3.5 |
| 1-menthol | 34.1 |
| pulegone | 1.9 |

TABLE 11
An Essential Oil Derived From a Hybrid Mint
Plant According to One Aspect Presented Herein.

| Component | % |
|---|---|
| l-linonene | 3.5 |
| 1,8-cineole | 6.1 |
| l-menthone | 8.7 |
| menthofuran | 18.2 |
| isomenthone | 0.7 |
| l-menthyl acetate | 7.1 |
| 1-menthol | 38.6 |
| pulegone | 1.1 |

TABLE 12
An Essential Oil Derived From a Hybrid Mint
Plant According to One Aspect Presented Herein.

| Component | % |
|---|---|
| l-linonene | 1.7 |
| 1,8-cineole | 4.9 |
| l-menthone | 18.5 |
| menthofuran | 6.7 |
| isomenthone | 2.4 |
| l-menthyl acetate | 6.5 |
| 1-menthol | 0.4 |
| pulegone | 1.3 |

TABLE 13
An Essential Oil Derived From a Hybrid Mint
Plant According to One Aspect Presented Herein.

| Component | % |
|---|---|
| l-linonene | 1.9 |
| 1,8-cineole | 1.1 |
| l-menthone | 61.7 |
| menthofuran | 1.4 |
| isomenthone | 4.0 |
| l-menthyl acetate | 1.2 |
| 1-menthol | 8.0 |
| pulegone | 0.6 |

TABLE 14
An Essential Oil Derived From a Commercial Mint
Plant According to One Aspect presented herein.

| Component | % |
|---|---|
| d-linonene | 10.5 |
| 1,8-cineole | 1.4 |
| 3-octanol | 1 |
| menthone | <1.0 |
| dihydrocarvone | 1.3 |
| l-carvone | 68.8 |

TABLE 15
An Essential Oil Derived From a Commercial Mint
Plant According to One Aspect presented herein.

| Component | % |
|---|---|
| d-linonene | 16.8 |
| 1,8-cineole | 1.3 |
| 3-octanol | 1.9 |
| menthone | 1.0 |
| dihydrocarvone | 0.9 |
| l-carvone | 69.2 |

TABLE 16
An Essential Oil Derived From a Commercial Mint
Plant According to One Aspect presented herein.

| Component | % |
|---|---|
| l-linonene | 1.78 |
| 1,8-cineole | 5.33 |
| l-menthone | 20.00 |
| menthofuran | 4.35 |
| isomenthone | 3.19 |
| l-menthyl acetate | 5.09 |
| neo-menthol | 5.40 |
| β-caryophyllene | 0.83 |
| 1-menthol | 45.20 |
| pulegone | 2.17 |
| germacrene-D | 1.22 |

TABLE 17
An Essential Oil Derived From a Commercial Mint
Plant According to One Aspect presented herein.

| Component | % |
|---|---|
| l-linonene | 1.73 |
| 1,8-cineole | 4.90 |
| l-menthone | 19.43 |
| menthofuran | 4.23 |
| isomenthone | 3.14 |
| l-menthyl acetate | 5.36 |
| neo-menthol | 5.52 |
| β-caryophyllene | 0.85 |
| 1-menthol | 44.60 |
| pulegone | 2.14 |
| germacrene-D | 1.37 |

TABLE 18
An Essential Oil Derived From a Commercial Mint
Plant According to One Aspect presented herein.

| Component | % |
|---|---|
| l-linonene | 3.0 |
| 1,8-cineole | <1.0 |
| l-menthone | 7.3 |
| menthofuran | 0.0 |
| isomenthone | 3.6 |
| l-menthyl acetate | 2.9 |
| 1-menthol | 73.3 |
| pulegone | 0.0 |

TABLE 19
A Composition According to One Aspect Presented Herein.

| Component | % |
|---|---|
| anethol | 9.3 |
| carvone gauche | 8.8 |
| freeze 10 ethyl acetate | 0.9 |
| menthol melted | 27.3 |
| menthol racemic | 5.5 |
| Mint arvensis terpeneless | 6.5 |
| 07-A3-5A | 2.5 |
| Mint Piperita Rectified India | 6.8 |
| Mint Spicata Native | 9.1 |
| Mint Spicata US | 10.4 |
| linseed oil | 12.9 |

TABLE 20
A Composition According to One Aspect Presented Herein.

| Component | % |
|---|---|
| Nova Mint Flavor | 1.2 |
| Freeze Bamboo Mint Flavor | 4 |
| anethol | 9.11 |
| decal | 0.2 |
| eucalyptol | 1.55 |
| eucalyptus globulus | 2.5 |
| eugenol | 0.84 |
| Freeze 10 ethyl acetate | 1.48 |
| HEDIONE FAB @ 10% Menth Bresil | 0.1 |
| allyle heptanoate | 0.3 |
| Menthe Bresil | 8.08 |
| menthol melted | 41.08 |
| menthol racemic | 7.3 |
| Mint Euromint | 0.95 |
| 07-A3-5A | 14.2 |
| Mint Piperita Cascade | 6.25 |
| octalactone G | 0.48 |
| Violette AI II @1% Mint arvensis terpeneless | 0.3 |

TABLE 21
A Composition According to One Aspect Presented Herein.

| Component | % |
|---|---|
| Rain Forest Mint Flavor | 1.21 |
| anethol | 10.5 |
| eucalyptus globulus | 3.92 |
| eugenol F | 0.45 |
| Freeze 10 ethyl acetate | 1.31 |
| glycyrrhizine ammoniacale @ 1% PG | 0.4 |
| menthol melted | 43.85 |
| menthol racemic | 3.41 |
| Mint Arvensis TPC | 8.74 |
| Mint Euromint | 0.55 |
| Mint Piperita ABS @10% Mint Rectified India | 0.02 |
| 07-A3-5A | 1 |
| Mint Piperita Cascade | 10.37 |
| Mint Piperita Rectified India | 12.27 |
| linseed oil | 2 |

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1. A composition comprising:
a. a mint composition, which comprises a combination selected from the group consisting of:
i. a first combination, which comprises l-carvone and d-limonene, wherein the weight ratio of the l-carvone to d-limonene in the first combination is from 0.83:1 to 1.25:1;
ii. a second combination, which comprises menthofuran and l-menthol, wherein the weight ratio of the menthofuran to l-menthol in the second combination is from 0.87:1 to 1.31:1;
iii. a third combination, which comprises l-menthol and l-menthyl acetate, wherein the weight ratio of the l-menthol to l-menthyl acetate in the third combination is from 6.15:1 to 9.23:1;
iv. a fourth combination, which comprises l-menthol and l-menthone, wherein the weight ratio of the l-menthol to l-menthone in the fourth combination is from 1.66:1 to 2.50:1;
v. a fifth combination, which comprises l-menthol and l-menthone, wherein the weight ratio of the l-menthol to l-menthone in fifth the combination is from 0.98:1 to 1.47:1;
vi. a sixth combination, which comprises l-menthol and l-menthone, wherein the weight ratio of the l-menthol to l-menthone in the sixth combination is from 2.52:1 to 3.77:1;
vii. a seventh combination, which comprises l-menthol and l-menthone, wherein the weight ratio of the l-menthol to l-menthone in the seventh combination is from 1.71:1 to 2.61:1;
viii. an eighth combination, which comprises l-menthol and l-menthone, wherein the weight ratio of the l-menthol to l-menthone in the eighth combination is from 1.73:1 to 2.60:1;
ix. a ninth combination, which comprises l-menthol and menthofuran, wherein the weight ratio of the l-menthol to menthofuran in the ninth combination is from 1.71:1 to 2.56:1;
x. a tenth combination, which comprises l-menthol and menthofuran, wherein the weight ratio of the l-menthol to menthofuran in the tenth combination is from 1.70:1 to 2.55:1;
xi. an eleventh combination, which comprises l-menthone and l-menthol, wherein the weight ratio of the l-menthone to l-menthol in the eleventh combination is from 0.95:1 to 1.43:1;
xii. a twelfth combination, which comprises l-menthone and l-menthol, wherein the weight ratio of the l-menthone to l-menthol in the twelfth combination is from 6.17:1 to 9.26:1; and
xiii. a thirteenth combination, which comprises menthofuran and l-menthyl acetate, wherein the weight ratio of the menthofuran to l-menthyl acetate in the thirteenth combination is from 1.92:1 to 2.89:1; and
b. at least one cooling compound, wherein the at least one cooling compound comprises 2-(4-methylphenoxy)-N-(1H-pyrazol-3-yl)-N-(2-thienylmethyl) acetamide.

2. The composition according to claim 1, wherein the mint composition is present in the flavor composition in an amount ranging from 0.1 to 100% by weight.

3-8. (canceled)

9. The composition according to claim 1, further comprising at least one extract of a commercial mint plant.

10. The composition according to claim 1, wherein the at least one cooling compound is in the flavor composition in an amount ranging from 0.005 to 0.4% by weight.

11. (canceled)

12. (canceled)

13. The composition according to claim 1, wherein the composition is a cosmetic preparation.

14. The composition according to claim 13, wherein the composition is an oral care preparation.

15. The composition according to claim 1, wherein the composition is a pharmaceutical preparation.

16. The composition according to claim 1, wherein the composition is a food preparation.

17. The composition according to claim 1, wherein the composition is a fragrance preparation.

18. The composition according to claim 1, wherein the at least one cooling compound further comprises: 2-isopropyl-N,2,3-trimethylbutyramide); N-ethyl-p-menthane-3-carboxamide; ethyl 3-(p-menthane-3-carboxamido)acetate; (1R,2S,5R)—N-(4-methoxyphenyl)-p-menthanecarboxamide; N-ethyl-2,2-diisopropylbutanamide; N-cyclopropyl-5-methyl-2-isopropylcyclohexanecarboxamide; N-(1,1-dimethyl-2-hydroxyethyl)-2,2-diethylbutanamide; N-(1,1-dimethyl-2-hydroxyethyl)2,2-diethylbutanamide; menthoxyethanol; N-(4-cyanomethyl-phenyl)-p-menthanecarboxamide; N-(2-(pyridin-2-yl)-ethyl)-3-p-menthanecarboxamide; N-(2-hydroxyethyl)-2-isopropyl-2,3-dimethylbutanamide; N-(4-(carbamoylmethyl)phenyl)-menthylcarboxamide; (1R,2S,5R)—N-(4-methoxyphenyl)-p-menthanecarboxamide; (2S,5R)—N-[4-(2-amino-2-oxoethyl)phenyl]-p-menthanecarboxamide; N-cyclopropyl-5-methyl-2-isopropylcyclohexanecarbonecarboxamide; 2-[(2-p-menthoxy)ethoxy]ethanol; 2,6-diethyl-5-isopropyl-2-methyltetrahydropyran; trans-4-tert-butylcyclohexanol; 2-(p-tolyloxy)-N-(1H-pyrazol-5-yl)-N-((thiophen-2-yl)methyl)acetamide; menthone glycerol ketal; menthone glycerol ketal; (−)-menthoxypropane-1,2-diol; 3-(l-menthoxy)-2-methylpropane-1,2-diol; isopulegol; (+)-cis p-menthane-3,8-diol; (−)-trans p-menthane-3,8-diol; 2,3-dihydroxy-p-menthane; 3,3,5-trimethylcyclohexanone glycerol ketal; menthyl pyrrolidone carboxylate; (1R,3R,4S)-3-menthyl-3,6-dioxaheptanoate; (1R,2S,5R)-3-menthyl methoxyacetate; (1R,2S,5R)-3-menthyl 3,6,9-trioxadecanoate; (1R,2S,5R)-3-menthyl 3,6,9-trioxadecanoate; (1R,2S,5R)-3-menthyl (2-hydroxyethoxy)acetate; (1R,2S,5R)-menthyl 11-hydroxy-3,6,9-trioxaundecanoate; cubebol; N-(4-cyanomethylphenyl) p-menthanecarboxamide; 2-isopropyl-5-methylcyclohexyl 4-(dimethylamino)-4-oxobutanoate; N-(4-cyanomethylphenyl) p-menthanecarboxamide, N-(2-pyridin-2-ylethyl) p-menthanecarboxamide; menthyl lactate; 6-isopropyl-3,9-dimethyl-1,4-dioxaspiro[4.5]decan-2-one; N-benzo[1,3] dioxol-5-yl-3-p-menthanecarboxamide; N-(1-isopropyl-1,2-dimethylpropyl)-1,3-benzodioxole-5-carboxamide; N—(R)-2oxo-tetrahydrofuran-3-yl-(1R,2S,5R)-p-menthane-3-carboxamide; 2,2,5,6,6-pentamethyl-2,3,6,6a-tetrahydropentalen-3a(1H)-ol; 5-(2-hydroxy-2-methylpropyl)-3,4,4-trimethylcyclopent-2-en-1-one; (1R,2S,5R)-2-isopropyl-5-methyl-N-(2-(pyridin-2-yl)ethyl)cyclohexanecarboxamide; (2S,5R)-2-isopropyl-5-methyl-N-(2-(pyridin-4-yl)ethyl)cyclohexanecarboxamide; N-(4-cyano-methylphenyl) p-menthanecarboxamide; (1S,2S,5R)—N-(4-(cyanomethyl)phenyl)-2-isopropyl-5-methylcyclohexanecarboxamide; 1/7-isopropyl-4/5-methyl-bicyclo[2.2.2]oct-5-ene derivatives; 4-methoxy-N-phenyl-N-[2-(pyridin-2-yl)ethyl]benzamide; 4-methoxy-N-phenyl-N-[2-(pyridin-2-yl)ethyl]benzenesulfonamide; 4-chloro-N-phenyl-N-[2-(pyridin-2-yl)ethyl]-benzenesulfonamide; 4-cyano-N-phenyl-N-[2-(pyridin-2-yl)ethyl]benzenesulfonamide; 4-((benzhydrylamino)methyl)-2-methoxyphenol; 4-((bis(4-methoxyphenyl)-methylamino)-methyl)-2-methoxyphenol; 4-((1,2-diphenylethylamino)methyl)-2-methoxyphenol; 4-((benzhydryloxy)methyl)-2-methoxyphenol; 4-((9H-fluoren-9-ylamino)methyl)-2-methoxyphenol; 4-((benzhydrylamino)methyl)-2-ethoxyphenol; 1-(4-methoxyphenyl)-2-(1-methyl-1H-benzo[d]imidazol-2-yl)vinyl4-methoxybenzoate; 2-(1-isopropyl-6-methyl-1H-benzo[d]imidazol-2-yl)-1-(4-methoxyphenyl)vinyl4-methoxybenzoate; (Z)-2-(1-isopropyl-5-methyl-1H-benzo[d]imidazol-2-yl)-1-(4-methoxy-phenyl)vinyl-4-methoxybenzoate; 3-alkyl-p-methan-3-ol derivatives; derivatives of fenchyl, D-bornyl, L-bornyl, exo-norbornyl, 2-methylisobornyl, 2-ethylfenchyl, 2-methylbornyl, cis-pinan-2-yl, verbanyl or isobornyl; menthyl oxamate derivatives; menthyl 3-oxocarboxylic acid esters; N alpha-(menthanecarbonyl)amino acid amides; p-menthane carboxamide; (−)-(1R,2R,4S)-dihydro-umbellulol; p-menthane alkyloxy amides; cyclohexane derivatives; butone derivatives; 3-menthoxy-1-propanol; 1-menthoxy-2-propanol; 1-[2-hydroxyphenyl]-4-[2-nitrophenyl-]-1,2,3,6-tetrahydropyrimidine-2-one; 4-methyl-3-(1-pyrrolidinyl)-2[5H]-furanone; or combinations thereof.
